(19) United States
(12) Patent Application Publication
(10) Pub. No.: US 2005/0261220 A1
(43) Pub. Date: Nov. 24, 2005

(54) Tumor treating combinations, compositions and methods

(76) Inventors: Geoffrey Wayne Krissansen, Auckland (NZ); Xueying Sun, Auckland (NZ); Jagat Rakesh Kanwar, Auckland (NZ)

Correspondence Address: FISH & RICHARDSON PC, P.O. BOX 1022, MINNEAPOLIS, MN, 55440-1022, US

(21) Appl. No.: 11/037,540

(22) Filed: Jan. 18, 2005

Related U.S. Application Data
(63) Continuation-in-part of application No. PCT/NZ03/00156, filed on Jul. 18, 2003.

(30) Foreign Application Priority Data
Jul. 19, 2002 (NZ) 520321

(51) Int. Cl. A61K 48/00

(52) U.S. Cl. 514/44

(57) ABSTRACT

The invention relates to compositions and methods of use in the treatment of tumors in animals. The invention is particularly concerned with the combination of HIF inhibiting agents, specifically antisense HIF-1, with antiangiogenic agents.

### RELATED APPLICATIONS

This application is a continuation-in-part of International Application Serial No. PCT/NZ03/000156, filed Jul. 18, 2003, which claims priority to New Zealand Application Serial No. 520321, filed Jul. 19, 2002, the contents of which are incorporated herein in their entirety.

### TECHNICAL FIELD

The invention relates to the treatment of tumors. The invention also relates to compositions and methods of use in such treatments.

### BACKGROUND

HIF-1 regulates cellular adaptation to changes in the oxygen availability by regulating genes involved in angiogenesis, erythropoiesis, energy and iron metabolism, tissue matrix metabolism, and cell survival decisions; which are key factors for tumor growth and survival (1-3). HIF-1 is an αβ heterodimer of which the β subunit is expressed constitutively and is not significantly affected by hypoxia, whereas levels of the α subunit rise markedly with hypoxia, and fall rapidly under normoxic conditions.

Von Hippel-Lindau (VHL) disease is an autosomal dominant familial cancer syndrome that predisposes affected individuals to a variety of highly vascular tumors (4, 5). The most common tumors are hemangioblastomas of the central nervous system, renal cell carcinoma (RCC), and pheochromocytoma. VHL kindreds have germline mutations in the VHL gene, and somatic inactivation or loss of the remaining wild-type VHL allele is linked to tumor formation.

VHL is a tumor suppressor, whose functional inactivation stimulates tumor formation in a variety of ways, in particular by increasing the stability of Hypoxia Inducible Factor-1 (HIF-1) (5, 6). A 35 amino acid subdomain of the a domain of the 30 kDa von Hippel-Lindau protein (PVHL) binds elongin C, which recruits additional proteins including elongin B, cullin-2, the RING-H2 protein Rbx1/Roc1, and ubiquitin conjugating enzyme E2, to form a ubiquinating complex. The β domain of pVHL binds hypoxia-inducible factor (HIF) α subunits HIF-1α and HIF-2α, targeting them for ubiquitination and proteasomal destruction in a VHL α-domain-dependent manner (7). The binding of HIF-1α subunits to VHL, and their rapid degradation by the VHL ubiquitinating complex under normoxic conditions, is regulated by oxygen and iron-dependent hydroxylation of Pro-564 within HIF-1α (8). Mutation of the α and β domains of VHL either prevents formation of a VHL ubiquitinating complex, and or binding to HIF-1, respectively, leading to stabilization of HIF-1 (3, 7). A hypoxic phenotype results in which increased levels of HIF-1 induce the synthesis of hypoxia-inducible genes such as vascular endothelial growth factor (VEGF), platelet derived growth factor, and glucose transporter-1 (Glut-1), which assist tumor growth by stimulating tumor angiogenesis, and metabolism (9-12).

Reintroduction of wild-type VHL into the VHL-negative tumor RCC in which both VHL alleles are either inactivated or lost, restores VHL-mediated functions, and leads to a loss of tumorigenicity in nude mice (13).

Endostatin is a 20 kDa C-terminal fragment of collagen XVIII, a member of a family of collagen-like proteins called multiplexins (14). Collagen XVIII is a component of the basement membrane zones that surround blood vessels (15). Endostatin is an inhibitor of angiogenesis. It specifically inhibits endothelial cell proliferation, that is, it has no effect on the growth of other cell types. It is produced naturally by a murine hemangioendothelioma, from which it was first purified (14). Recombinant E. coli-derived endostatin, when added at a site remote from the primary tumor, has a systemic effect causing even very large tumors (1% of body weight) to regress to dormant microscopic nodules (14). Hence tumors can be forced to regress over 150-fold in size to less than 1 mm³. As long as treatment is continued there is no tumor regrowth, and no toxicity. When treatment is initially stopped tumors regrow, however treatment can be continued and drug-resistance does not develop over multiple treatment cycles (16). Remarkably, repeated cycles of antiangiogenic therapy were followed by self-sustained dormancy that remained for the lifetime of most animals (16). The mechanism for the persistence of tumor dormancy after therapy is suspended is unknown, but it is not due to an antitumor immune response, as tumors injected at sites remote from the treated tumor grew unchecked. The dormant tumors which are of a size that can survive without blood vessels display no net gain in size due to a balance between high proliferation of tumor cells, and high apoptosis.

The mechanism of action of endostatin remains unknown. The anti-angiogenic effects of endostatin may be due in part to its ability to block the attachment of endothelial cells to fibronectin via α5β1, and αVβ3 integrins (17), and/or α2β1 (18).

Vascular endothelial growth factor (VEGF) is a major cytokine known to induce tumor angiogenesis. Vascular endothelial growth factor (VEGF) binding to the kinase domain receptor (KDR/FLK1 or VEGFR-2) mediates vascularization and tumor-induced angiogenesis. A synthetic peptide, ATWLPPR has been shown to abolish VEGF binding to cell-displayed KDR, and abolished VEGF-induced angiogenesis in a rabbit corneal model (19).

Angiostatin is a 38,000-Mr protein comprising the first four of five highly homologous 80-amino acid residue long triple-loop structures termed kringles.⁷⁵ It can inhibit the growth of a broad array of murine and human tumors established in mice,⁷⁶ and is non-toxic such that tumors can be subjected to repeated treatment cycles, without exhibiting acquired resistance to therapy.⁷⁷ Its tumor-suppressor activity may arise from its ability to inhibit the proliferation of endothelial cells by binding to the α/β-subunits of ATP synthase,⁷⁸ by inducing apoptotic cell death,⁷⁹ by subverting adhesion plaque formation and thereby inhibiting the migration and tube formation of endothelial cells,⁸⁰ and/or by down-regulating vascular endothelial growth factor (VEGF) expression.^{81,82} Angiostatin reduces the phosphorylation of the mitogen-activated protein kinases ERK-1 and ERK-2 in human dermal microvascular cells in response to VEGF.⁸³ Endothelial progenitor cells are exquisitively sensitive to the effects of angiostatin, and may be the most important target of angiostatin.⁸⁴ Gene transfer of angiostatin into small solid EL-4 lymphomas established in mice led to reduced tumor angiogenesis, and weak inhibition tumor growth.⁸⁵ In contrast, when angiostatin gene therapy was preceded by in situ gene transfer of the T cell costimulator B7-1, large tumors were rapidly and completely eradicated; whereas B7-1 and angiostatin monotherapies were ineffective. Gene transfer of AAV-angiostatin via the portal vein led to significant suppression of the growth of both nodular and, metastatic EL-4 lymphoma tumours established in the liver, and prolonged the survival time of the mice.⁸⁶ Survivin is a recently identified member of the inhibitor of apoptosis (IAP) proteins⁵¹ which are now regarded as important targets in cancer therapy. Antisense complementary DNA (cDNA) and oligonucleotides that reduce the expression of the IAP protein Bcl-2 inhibit the growth of certain tumor cell lines in vitro.⁵¹⁻⁵³ Similarly, antisense oligonucleotides that reduce survivin expression in tumors cells induce apoptosis and polyploidy, decrease colony formation in soft agar, and sensitize tumor cells to chemotherapy in vitro.⁵⁴⁻⁵⁷ Intratumoral injection of plasmids that block survivin expression were found to inhibit tumor growth, particularly the growth of large tumors.⁵⁸

Survivin is highly expressed in newly formed blood vessels in response to vascular endothelial growth factor and basic fibroblast growth factor,^{59,60} and mediates angiopoietin inhibition of endothelial cell apoptosis.⁶¹ Survivin promotes a novel mechanism of endothelial cell drug “resistance”, since angiogenic factors that induce the expression of survivin may act to shield tumor endothelial cells from the apoptotic effects of chemotherapy.⁶² In accord, antisense survivin facilitated endothelial cell apoptosis and promoted vascular regression during tumor angiogenesis.^{63,64}

The development and growth of tumors is complex. Despite any positive results in tumor treatment described to date, there would be distinct advantages in providing alternative options, including being able to provide combinations of active agents which contribute to the options available for tumor treatment.

Bibliographic details of the publications referred to herein are collected at the end of the description.

### SUMMARY OF THE INVENTION

The inventors have surprisingly discovered that if the administration of antisense HIF-1 is combined with that of an appropriate antiangiogenic agent, tumor cell apoptosis may be enhanced, tumor angiogenesis inhibited, and tumors may be more effectively treated.

Accordingly, in a first aspect of the present invention there is provided a method of treating tumors in a mammal, the method comprising at least the step of administering an effective amount of a HIF inhibiting agent together with an effective amount of at least one antiangiogenic agent.

Preferably the present invention provides a method of treating tumors in a mammal, the method comprising at least the step of administering an effective amount of antisense HIF-1 together with an effective amount of at least one antiangiogenic agent.

Preferably, the antisense HIF-1 is antisense HIF-1α.

Preferably the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

Preferably antisense HIF-1α is provided by a vector adapted to produce antisense HIF-1α in use. Preferably, the vector is a nucleic acid vector. Alternatively, the vector is a viral vector comprising nucleic acid in a viral capsid.

Preferably an agent capable of increasing VHL in a tumor is a vector adapted to express VHL in use. Preferably, the vector is a nucleic acid vector. Alternatively, the vector is a viral vector comprising nucleic acid in a viral capsid.

Preferably, an agent capable of increasing VHL is one adapted to over-express native VHL within the tumor.

Preferably antisense survivin is provided by a vector adapted to produce antisense antisense survivin in use. Preferably, the vector is a nucleic acid vector. Alternatively, the vector is a viral vector comprising nucleic acid in a viral capsid.

Preferably, VEGF blocking peptide is provided by a vector adapted to express VEGF blocking peptide in use. Preferably, the vector is a nucleic acid vector. Alternatively, the vector is a viral vector comprising nucleic acid in a viral capsid.

Preferably, angiostatin is provided by a vector adapted to express angiostatin in use. Preferably, the vector is a nucleic acid vector. Alternatively, the vector is a viral vector comprising nucleic acid in a viral capsid.

Preferably, endostatin is provided by a vector adapted to express endostatin in use. Preferably, the vector is a nucleic acid vector. Alternatively, the vector is a viral vector comprising nucleic acid in a viral capsid.

Preferably the antisense HIF-1α and one or more antiangiogenic agents are administered intratumorally. Alternatively, the agents are administered intraperitoneally, parenterally, or systemically.

Preferably the antisense HIF-1α and one or more antiangiogenic agents are coadministered. Alternatively, the antisense HIF-1α and one or more antiangiogenic agents are administered sequentially, in any order.

In another aspect, the invention provides a method of treating a tumor in an animal comprising at least the step of administering to said animal antisense HIF-1α with endostatin and/or VEGF blocking protein.

Preferably, antisense HIF-1α is administered in the form of a vector adapted to produce antisense HIF-1α in use. Preferably, the endostatin and/or VEGF blocking protein are administered to the mammal subcutaneously. Preferably the VEGF blocking protein and endostatin are co-administered subcutaneously. Preferably the administration of antisense HIF-1α and the co-administration of endostatin and VEGF blocking protein, proceed sequentially.

In another aspect, the invention provides a method of treating a tumor in an animal comprising at least the steps of administering to said animal antisense HIF-1α and over-expressing VHL in the tumor.

Preferably, over-expression of VHL occurs via administering a vector adapted to express VHL in use. Preferably, antisense HIF-1α is administered in the form of a vector adapted to produce antisense HIF-1α in use. Preferably, administration of the vector adapted to express VHL occurs first, followed by administration of the vector adapted to express antisense HIF-1α in use.

In another aspect, the invention provides a method of treating a tumor in an animal comprising at least the steps of administering to said animal antisense HIF-1α and angiostatin.

Preferably, antisense HIF-1α is administered in the form of a vector adapted to produce antisense HIF-1α in use. Preferably, angiostatin is administered in the form of a vector adapted to express angiostatin in use. Preferably, administration of the vector adapted to express angiostatin in use is administered first, followed by administration of vector adapted to produce antisense HIF-1α in use.

In another aspect, the invention provides a method of treating a tumor in an animal comprising at least the steps of administering to said animal antisense HIF-1α and antisense survivin.

Preferably, antisense HIF-1α and antisense survivin are administered in the form of a vector adapted to produce antisense HIF-1α in use.

In another aspect, the present invention provides a method of enhancing tumor cell apoptosis in an animal, the method comprising at least the step of administering an effective amount of antisense HIF-1α together with an effective amount of at least one antiangiogenic agent.

Preferably the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

In another aspect, the present invention provides a method of inhibiting tumor angiogenesis in an animal, the method comprising at least the step of administering an effective amount of antisense HIF-1α together with an effective amount of at least one antiangiogenic agent. Preferably the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

In another aspect, the invention provides a composition comprising antisense HIF-1α, or a vector adapted to produce antisense HIF-1α in use, together with one or more antiangiogenic agents and optionally one or more pharmaceutically acceptable excipients and/or carriers.

Preferably the antiangiogeneic agents are selected from the group comprising endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

Preferably, the composition is suitable for intratumoral administration. Alternatively, the composition is suitable for intraperitoneal administration. Alternatively, the composition is suitable for systemic administration. Preferably, the composition is suitable for subcutaneous administration.

A tumor treating composition combining (i) antisense HIF-1α, or a vector adapted to produce antisense HIF-1α, and (ii) one or more antiangiogenic agents, wherein the combination of (i) and (ii) is adapted for sequential administration to a mammal.

The use of antisense HIF-1α, or a vector adapted to produce antisense HIF-1α, and one or more antiangiogenic agent in the manufacture of a medicament for enhancing tumor cell apoptosis, inhibiting tumor angiogenesis, or for tumor treatment in an animal.

Preferably, the antiangiogenic agent is selected from the group comprising endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

In another aspect, the invention may be seen to provide a method of systemically treating tumors in a mammal, comprising at least, in any order, the steps of:
(a) administering a systemically effective amount of an HIF-1 inhibiting agent and
(b) administering a systemically effective amount of an antiangiogenic agent.

Preferably the HIF-1 inhibiting agent and the antiangiogenic agent are administered by subcutaneous injection, together with suitable carriers and/or excipients.

Preferably the HIF-1 inhibiting agent that is subcutaneously administered is selected from any one or more of HIF-1 antagonists including cellular ligands, and cell permeable agents that antagonises HIF-1 expression and function such as cell-permeable VHL, cell-permeable dominant-negative HIF-1 peptides, and antisense HIF-1 polynucleotides.

Preferably the antiangiogenic agent that is subcutaneously administered is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

Preferably step (a) and step (b) are separate sequential steps in any order.

Preferably step (a) and step (b) are unitary and the agents are co-administered.

### DRAWINGS

These and other aspects of the present invention, which should be considered in all its novel aspects, will become apparent from the following description, which is given by way of example only, with reference to the accompanying figures:

FIGS. 1A-E Intratumoral injection of expression plasmids encoding VHL and antisense HIF-1α downregulates HIF-1α and VEGF in tumors. (A) Immunohistochemistry to analyze the expression of plasmids injected into tumors. Tumors of 0.4 cm diameter were injected with empty pcDNA3 vector (pcDNA3), or expression plasmids encoding either VHL, antisense HIF-1α (aHIF), or a combination of VHL and antisense HIF-1α (VHL+aHIF). Tumor sections prepared two days after plasmid injection were stained (brown) for VHL with the rabbit polyclonal anti-VHL antibody FL-181. Magnification, ×100. (B) Over-expression of VHL by intratumoral injection of a VHL expression plasmid downregulates HIF-1α. EL-4 tumors as in (A) were stained with the mouse anti-mouse HIF-1α mAb H1α67. Magnification, ×100. (C) Over-expression of VHL by intratumoral injection of a VHL expression plasmid downregulates VEGF expression. EL-4 tumor sections as in (A), but prepared 4 days after plasmid injection, were stained with the Ab-1 rabbit polyclonal antibody against VEGF. Magnification, ×100. (D) Western blot analysis of homogenates of tumor cells extracted from tumors. Tumor cell homogenates prepared from tumors as in (A) were injected with empty plasmid (lane 1), or VHL (lane 2) and antisense HIF-1α (lane 3) plasmids, or a combination of VHL and antisense HIF-1α plasmids (lane 4). They were resolved by SDS-PAGE, and Western blotted with antibodies against VHL and HIF-1α, and VEGF as indicated. (E) Decrease in the percentage of HIF-1α positive-staining cells after injection of VHL plasmids. The numbers of HIF-1α positive cells in sections (×40 magnification) illustrated in (B) were counted in 10 blindly chosen random fields. n, number of tumors assessed. There was a significant (P<0.01) difference in the numbers of HIF-1α positive cells in sections of tumors injected with empty pcDNA3 plasmid versus tumors injected with VHL plasmid.

FIGS. 2A-B Intratumoral injection of a combination of VHL and antisense HIF-1α plasmids eradicates large EL-4 tumors, whereas monotherapies are only effective against small tumors. (A) Intratumoral injection of a combination of VHL and antisense HIF-1α plasmids eradicates large EL-4 tumors. Tumors 0.4 cm in diameter were injected at day 0 with expression plasmids encoding VHL, antisense HIF-1α, or empty vector (Control), or with a combination of VHL expression plasmid, followed 48 h later by injection of antisense HIF-1α plasmid. Tumor size was recorded for 15 days. Complete tumor regression is denoted by a vertical arrow. Mice were euthanased when tumors reached 1 cm in diameter (denoted by stars). (B) Increased dosages of VHL plasmid fail to eradicate large tumors. Tumors 0.4 cm in diameter were injected with dosages of VHL plasmid ranging from 100 to 250 μg. Tumor size was recorded for 15 days. All the mice were euthanased when tumors reached 1 cm.

FIGS. 3A-B Intratumoral injection of expression plasmids encoding VHL and antisense HIF-1α inhibits tumor angiogenesis. (A) Illustrated are sections prepared from 0.4 cm tumors injected 4 days earlier with empty pcDNA3 vector (pcDNA3), or expression plasmids encoding either VHL, antisense HIF-1α (aHIF), or a combination of VHL and antisense HIF-1α (VHL+aHIF). Sections were stained with anti-CD31 antibody MEC13.3 to visualize blood vessels. (B) Measurement of tumor vascularity. Tumor blood vessels stained with the anti-CD31 mAb were counted in 5 blindly chosen random fields to record mean blood vessel counts per section (40× magnification field). n, number of tumors assessed. A significant (P<0.01) difference in mean vessel counts between tumors injected with therapeutic plasmid vectors versus tumors injected with empty pcDNA3 plasmid is donated by stars.

FIGS. 4A-B Intratumoral injection of expression plasmids encoding VHL and antisense HIF-1α enhances tumor cell apoptosis. (A) Tumor sections were prepared from 0.4 cm diameter tumors injected 4 days earlier with either empty pCDNA3 vector, or plasmids encoding VHL, anti-sense HIF-1α (aHIF), or a combination of VHL and anti-sense HIF-1α. Tumor sections were stained by TUNEL analysis for apoptotic cells (here colored grey). Magnification ×100. (B) TUNEL positive cells were counted to record the apoptosis index (AI) (40× magnification field). n, number of tumors assessed.

FIGS. 5A-C Antisense HIF-1α synergizes with endostatin and VEGF blocking peptide to eradicate large tumours. Mice bearing large tumours (0.5 cm in diameter) received (A) intratumoral (IT) or (B) subcutaneous (SC) injections of either VEGF blocking peptide (30 mg/kg body weight) or endostatin (50 mg/kg of body weight). Tumors in another group of mice were injected intratumorally with 100 μg of antisense (AS) HIF-1α expression plasmid, or 100 μg of empty control plasmid (PLASMID). For combination therapy, antisense HIF-1α plasmid was injected into tumors 24 h after the VEGF blocking peptide (L-isomer only) and endostatin had been administered. Day 0 refers to the day the first reagent was administered. Tumor size was monitored for 70 days, and animals were killed when their tumors became larger than 1 cm in diameter. (C) Anti-angiogenic therapy fails to generate acquired immunity. Mice cured of their tumors were challenged with 2×10⁵ parental EL-4 cells injected into the opposing flank one or two weeks after disappearance of tumors (open arrow) and monitored for tumor re-growth for an additional 35 days.

FIGS. 6A-D Tumors rapidly become resistant to angiostatin treatment by upregulating the hypoxia-inducible pathway. (A, B) Intratumoral injection of angiostatin plasmid initially suppresses tumor growth, but subsequently results in accelerated growth. EL-4 tumors, approximately 0.1 (A), and 0.4 (B) cm in diameter, were injected at day 0 with expression plasmids encoding angiostatin, or empty plasmid. Tumor size was recorded until tumors reached 1 cm in diameter when mice were euthanased. (C) Immunohistochemical analysis of expression of plasmids and hypoxia-related proteins. Tumors of 0.4 cm diameter were examined 0, 4, and 7 d after injection with angiostatin expression vector, as indicated. They were sectioned and stained (brown-dark blue) for angiostatin with a mAb recognizing kringles 1-3 of plasminogen, for HIF-1α with the mouse anti-mouse HIF-1α mAb H1α67, and for VEGF with the Ab-1 rabbit polyclonal antibody against VEGF, as indicated. Magnification, ×100. (D) Western blot analysis of expression of plasmids and hypoxia-related proteins. Tumors were homogenized at 0 (lane 1), 4 (lane 2), and 7 (lane 3) d following injection of angiostatin. Homogenates were resolved by SDS-PAGE, and Western blotted with antibodies against angiostatin, HIF-1α, or VEGF, as indicated.

FIGS. 7A-C Intratumoral injection of antisense HIF-1α downregulates tumor angiogenesis and survival factors resulting in the eradication of small tumors and growth suppression of large tumors. (A, B) EL-4 tumors, approximately 0.1 (A) and 0.4 (B) cm in diameter, were injected at day 0 with expression plasmids encoding antisense HIF-1α, or empty vector. Tumor size was recorded until tumors reached 1 cm in diameter, when mice were euthanased. (C) Western blot analysis of expression of plasmids and hypoxia-related proteins. Tumors were homogenized 2 d after injection of empty vector (lane 1) and antisense HIF-1α plasmid (lane 2). Homogenates were resolved by SDS-PAGE, and Western blotted with antibodies against HIF-1α, VEGF, Glut-1, LDHA, and tubulin, which served as an internal control.

FIGS. 8A-B Combined antisense HIF-1α and angiostatin therapy eradicates large tumors and prevents acquired tumor resistance to angiostatin. (A) intratumoral injection of angiostatin and antisense HIF-1α eradicates large EL-4 tumors. EL-4 tumors, approximately 0.4 cm in diameter, were injected at day 0 with expression plasmids encoding either angiostatin, antisense HIF-1α, or a combination of angiostatin and antisense HIF-1α. Control tumors were injected with empty vector. Tumor size was recorded until tumors reached 1 cm in diameter, when mice were euthanased (denoted by vertical arrows). Complete tumor regression is denoted by stars. (B) Western blot analysis of expression of plasmids and hypoxia-related proteins. Tumors were homogenized 0 (lane 1), 4 (lane 2), and 10 (lane 3) d following combination therapy. Homogenates were resolved by SDS-PAGE, and Western blotted with antibodies against HIF-1α, VEGF, Glut1, LDHA, and tubulin, which served as an internal control.

FIGS. 9A-C Antisense HIF-1α synergizes with angiostatin to inhibit tumor angiogenesis. (A) Illustrated are sections prepared from tumors 0.4 cm in diameter injected 0, 4 and 10 d earlier with angiostatin, and angiostatin plus antisense HIF-1α plasmids, as indicated. Sections were stained with the anti-CD31 mAb MEC13.3 to visualize blood vessels. (B, C) Measurement of tumor vascularity. (B) Tumor blood vessels stained with the anti-CD31 mAb were counted in 5 blindly chosen random fields to record mean blood vessel counts per section (40× magnification). (C) Histograms showing the median centile distances (±SD) to the nearest CD31-labeled venules from an array of points within tumors that had been injected 4 and 10 d earlier with either angiostatin plasmid, or a combination of angiostatin and antisense HIF-1α plasmid. Tumors receiving empty vector served as controls. n, number of tumors assessed. Significant or highly significant differences in mean vessel counts, or median distances to the nearest CD31-stained vessels, compared with that in control groups are denoted by an asterisk (P<0.01), or two asterisks (P<0.001), respectively.

FIGS. 10A-B Antisense HIF-1α synergizes with angiostatin to enhance tumor cell apoptosis. (A) Sections prepared from 0.4 cm diameter tumors that had been injected 0, 4, and 10 d earlier with either angiostatin, or a combination of angiostatin and antisense HIF-1α were stained by TUNEL analysis for apoptotic cells (coloured green). Magnification ×100. (B) TUNEL positive cells were counted to record the Apoptosis Index (AI) (40× magnification). n, number of tumors assessed. Significant and highly significant differences in the AI, compared with that for control tumors, are donated by an asterisk (P<0.01), or two asterisks (P<0.001), respectively.

### DETAILED DESCRIPTION

The present invention is generally directed to compositions and methods for inhibiting tumor angiogenesis, enhancing tumor cell apoptosis and generally treating tumors (e.g., lymphoma and glioma) in animals. The approach taken by the inventors has been to determine whether HIF-1 inhibiting agents, particularly antisense HIF-1α, that targets a tumor and its ability to induce blood vessel formation, synergizes with antiangiogenic agents, such as endostatin, VHL, antiostatin, antisense survivin, and/or VEGF blocking peptide therapies that target the tumor vasculature.

Taken together, the results obtained by the inventors suggest a surprising synergism between HIF-1 inhibiting agents, particularly antisense HIF-1α, and antiangiogenic agents, and that therapies which involve administration of combinations of these agents may be beneficial in the treatment of cancer. Taken individually, the surprising synergisms between individual agents tested provide a number of unforeseen options for the treatment of tumors or cancers.

It has been found that engineered over-expression of VHL in tumors coupled with anti-sense HIF-1 treatment, produced a synergistic effect on solid vascular tumors. In particular this effect was seen in large solid vascular tumors.

It was also found that antisense HIF-1α synergizes with VEGF blocking protein and/or also with endostatin to target tumors. In addition a triple therapy of antisense HIF-1α, VEGF blocking protein, and endostatin was surprisingly effective using a systemic administration approach when VEGF blocking protein and endostatin were administered subcutaneously.

Further the inventors have surprisingly found synergies between antisense HIF-1α when combined with angiostatin.

As used herein, the term “vascular tumor” should not be taken to imply that such tumors are highly vascular.

As used in relation to the invention, the term “treating” or “treatment” and the like should be taken broadly. They should not be taken to imply that an animal is treated to total recovery. Accordingly, these terms include amelioration of the symptoms or severity of a particular condition or preventing or otherwise reducing the risk of further development of a particular condition.

An “effective amount” of an agent of use in a method of the invention, is an amount necessary to at least partly attain a desired response.

It should be appreciated that methods of the invention may be applicable to various species of animal, preferably mammals, more preferably humans.

The present invention is directed to exploring the use of HIF-1 inhibiting agents in combination treatments. The most preferable agent is one which produces antisense HIF-1α. As will be readily apparent a number of other agents may also have the effect of inhibiting HIF-1. These include VHL and other proteins, such as p53, or drugs that affect HIF-1 protein stability.

Inhibitors of HIF-1 stimulators/co-receptors (Jab1, p300, SRC-1, Ref-1) will also inhibit HIF-1 function. Others include peptide fragments of HIF-1 that act as dominant-negative inhibitors, pharmaceutical drugs based on the sequences of HIF-1 that inhibit HIF-1 function, nucleotides that mimic hypoxia response elements and disrupt the binding or interaction of HIF-1 with gene promoters, and drugs that inhibit transcription of the HIF-1 gene, or HIF-1-mediated transcription, among others.

As this application envisages the use of HIF inhibiting agent together with inter alia, VHL (as VHL also has an antiangiogenic effect), reference to the use of VHL as a monotherapy HIF-1 inhibiting agent is excluded from the definition of such agents for the purposes of this application. The use of over-expressed VHL as a monotherapy in a tumor is covered in a corresponding application to the same applicant.

In a particularly preferred embodiment, the HIF-1 inhibiting agents are antisense HIF-1α oligonucleotides or nucleic acid vectors adapted to produce antisense HIF-1α in use. An example of a suitable vector is provided hereinafter under the heading “Examples”. Persons of general skill in the art to which the invention relates will readily appreciate alternative nucleic acid vectors of use in the invention. For example, other naked plasmids that employ CMV promoters may be used.

Such vectors may be constructed according to standard techniques and/or manufacturers instructions, having regard to the published nucleic acid sequence of HIF-1α (GenBank accession number for human HIF-1 α is U22431, and the murine HIF-1 α accession number is AF003695). A specific example of how such a vector may be constructed is provided herein after under the heading “Methods”.

Viral vectors, comprising nucleic acid within a viral capsid, may also be suitable as agents adapted to produce antisense HIF-1α Suitable viral vectors include adenoviruses, adeno-associated virus (AAV) and lentiviruses however skilled persons may readily recognise other suitable viral vectors. One advantage of using such viral vectors is that they may allow for systemic administration, as opposed to localised administration to a tumour.

As mentioned above, the present invention is also directed to the use of antiangiogenic agents in combination treatments (ie with HIF-1 inhibiting agents). To that end, a number of antiangiogenic agents have been used in the experimental section. These include endostatin, angiostatin, antisense survivin, VEGF blocking peptide and VHL, alone and in combination. The inventors contemplate the use of other antiangiogenic agents which may be referred to herein after, or as may be known by persons skilled in the art to which the invention relates.

Nucleic acid or viral vectors may also be suitable for providing antisense survivin in a method of the invention. Further they are applicable to the provision of VEGF blocking protein, angiostatin, endostatin, and VHL to a tumor. For example, vectors may be constructed to allow for expression of these agents in use. Skilled persons will readily appreciate means for constructing such appropriate vectors having regard to the information herein, the published nucleic acid and/or amino acid sequences of relevance to the agents (GenBank accession numbers: VEGF blocking peptide, human M32977, AF022375, AY047581, and murine M95200; endostatin, human NM_{—}030582, murine NM009929; angiostatin, human M74220, AY192161, murine J04766; VHL, human AF010238, murine AF513984; and, survivin, human NM_{—}001168, murine NM_{—}009689).

It should be appreciated that nucleic acid vectors of use in the invention may include various regulatory sequences. For example, they may include tissue specific promoters, inducible or constitutive promoters. Further, they may include enhancers and the like which may aid in increasing expression in certain circumstances. Persons of general skill in the art to which the invention relates may appreciate various other regulatory regions which may provide benefit.

As mentioned above, suitable viral vectors of use in the invention are adenoviruses, adeno-associated virus and lentivirus. These may be constructed according to standard procedures in the art or in accordance with manufacturers instructions; for example see Xu, R., Sun, X., Chan, D., Li, H., Tse, L-Y., Xu, S., Xiao, W., Kung, H., Krissansen, G. W., and Fan, S-T. Long-term expression of angiostatin suppresses metastatic liver cancer in mice. Hepatol. 37:1451-60, 2003.

It will be appreciated that viral vectors will generally be attenuated such that they do not possess their original virulence.

Methods of the invention may involve the over-expression of VHL in a tumor. The term “over-expression” should be taken to refer to an increase in VHL expression above the baseline expression level for a particular tumor. “Over-expression” may occur by increasing expression from an endogenous VHL gene (ie that native to the tumor, or to surrounding tissue) or via introduction of a VHL-expressing transgene (as has been described above in relation to providing vectors adapted to express VHL in use).

The inventors also contemplate methods involving the administration of agents adapted to mimic the function of VHL (ie VHL mimetics), or to up-regulate such agents within the tumor.

Agents which may be suitable to stimulate endogenous VHL expression including those that stimulate VHL gene transcription, translation, or protein stability include “nonselective” (indomethacin) and COX-2-selective (NS-398) non steroidal anti-inflammatory drugs (NSAIDs)” (20). Skilled persons may appreciate other appropriate agents.

Reagents that mimic the effects of VHL would include drugs that interact with VHL effectors, and stimulate a response similar to that of VHL. Peptides and pharmaceutical type reagents based on the VHL protein sequence or structure could be used as VHL mimetics. Where such agents can be administered subcutaneously this mode of administration may be used.

While the inventors have exemplified the use of VEGF blocking peptide in a method of the invention, they contemplate that a mimetic of this peptide, or any other agent capable of blocking the expression or function of VEGF may be suitably used. For example, antisense oligonucleotides, antibodies, dominant negative peptides and pharmaceutical drugs may be suitable.

In addition, while the use of antisense survivin is explicitly exemplified, the inventors contemplate other agents capable of blocking the expression or function of survivin to be of use in the invention. For example, antibodies, dominant negative peptides and pharmaceutical drugs may be suitable.

While agents of use in the invention may be provided in the form of nucleic acid or viral vectors adapted in use to express or produce the specific agents it should also be appreciated that they may be provided as nucleic acids (in a vector or as oligonucletides) or proteins as is appropriate. For example, antisense oligonucleotides may be used. In addition, endostatin, VHL, angiostatin, and VEGF blocking peptide, for example, may simply be administered to an animal as peptides.

It should be appreciated that agents or compounds of use in the invention may be modified to assist their function in vivo for example by reducing their immunogenicity or increasing their lifetime in vivo. Agents may be modified (for example by addition of a carrier peptide or membrane translocating motif as will be known in the art (for example, Chariot™ peptide; Active Motif, Carlsbad, Calif., USA)) or formulated with additional agents to allow for their cell permeability and the like, as is mentioned further herein after. Persons of ordinary skill in the art to which the invention relates will readily appreciate appropriate modifications. However, by way of example, the agents may be PEGylated to increase their lifetime in vivo, based on, e.g., the conjugate technology described in WO 95/32003.

Administration of agents of use in methods of the invention may occur by any means known in the art, having regard to the nature of the agent to be administered. Such methods include intratumoral (IT) administration, or alternatively direct injection into blood vessels supplying the tumor could occur. Systemic administration may also be appropriate. The inventors have also demonstrated efficacy using intraperitoneal (IP) administration. In addition administration may be by way of injection into blood vessels directly supplying a tumor. Specific examples of administration routes of use for a particular agent are detailed herein after under the section “Examples”. However, it should be appreciated that the examples are not intended to limit the means by which a particular agent can be administered.

By way of general example, modes of administration may include oral, topical, systemic (eg. transdermal, intranasal, or by suppository), parenteral (eg. intramuscular, subcutaneous, or intravenous injection), intratumoral (eg. by injection, using ballistics); by implantation, and by infusion through such devices as osmotic pumps, transdermal patches, and the like.

IT and IP administration may occur via injection (as exemplified herein after) or any other method as may be readily known in the art to which the invention relates. Systemic administration may occur by any standard means. However, by way of example where viral vectors are used, they may be administered orally, subcutaneously, intravenously and intrarectally. Agents such as endostatin, angiostatin, and VEGF bocking protein may be administered subcutaneously, for example.

Persons of general skill in the art to which the invention relates will be able to readily appreciate the most suitable mode of administration having regard to the therapeutic agent to be used.

While compounds or agents of use in the invention may be administered alone, in general, they will be administered as pharmaceutical compositions in association with at least one or more carriers and/or excipients. Accordingly, compounds may be administered as naked DNAs, or using virus technologies, or as recombinant proteins, peptides, or pharmaceutical compositions, or by other means that any person of ordinary skill in the art would be able to devise.

Compositions may take the form of any standard known dosage form including tablets, pills, capsules, semisolids, powders, sustained release formulation, solutions, suspensions, elixirs, aerosols, liquids for injection, or any other appropriate compositions. Persons of ordinary skill in the art to which the invention relates will readily appreciate the most appropriate dosage form having regard to the nature of the tumor to be treated and the active agents to be used without any undue experimentation. It should be appreciated that one or more active agents described herein may be formulated into a single composition.

Compounds or agents compatible with this invention might suitably be administered by a sustained-release system. Suitable examples of sustained-release compositions include semi-permeable polymer matrices in the form of shaped articles, e.g., films, or microcapsules.

Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing the compound are prepared by methods known per se: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy.

Suitable carriers and/or excipients will be readily appreciated by persons of ordinary skill in the art, having regard to the nature of the agent to be formulated. However, by way of example, suitable liquid carriers, especially for injectable solutions, include water, aqueous saline solution, aqueous dextrose solution, and the like, with isotonic solutions being preferred for intravenous, intraspinal, and intracistemal administration and vehicles such as liposomes being also especially suitable for administration of agents, such as naked nucleic acid vectors to tumors.

In addition to standard diluents, carriers and/or excipients, compositions of the invention may be formulated with additional constituents, or in such a manner, so as to decrease the immunogenicity of an agent to be administered, or help protect its integrity and prevent in vivo degradation, for example. Persons of ordinary skill in the art to which the invention relates will readily appreciate constituents and techniques to this end.

Further agents of use in the invention may be modified, or formulated with suitable carriers, such that they are rendered cell permeable. This would have the advantage of aiding in the likes of systemic administration and subcutaneous administration. In the case of HIF-1 and survivin inhibiting agents one could use cellular ligands and cell permeable agents that antagonise expression and function of these proteins. These will include cell permeable dominant negative HIF-1 and survivin peptides, and antisense HIF-1 and survivin polynucleotides, amongst others as will be known in the art. For VHL this would include cell-permeable agents that stimulate VHL function or expression. Proteins may be made cell-permeable by conjugating to or missing with cell permeable peptides (for example, Chariot™ agent, as herein before mentioned).

The compositions may be formulated in accordance with standard techniques as may be found in such standard references as Gennaro A R: Remington: The Science and Practice of Pharmacy, 20^{th} ed., Lippincott, Williams & Wilkins, 2000, for example.

The amount of a compound in the composition may vary widely depending on the type of composition, size of a unit dosage, kind of excipients, and other factors well known to those of ordinary skill in the art. The combination of compounds could be provided to a user in a chemotherapeutic pack for ease of use and access. The pack could be constructed in any suitable manner as would be known to the skilled person.

As will be appreciated, the dose of an agent or composition administered, the period of administration, and the general administration regime may differ between subjects depending on such variables as the severity of symptoms, the type of tumor to be treated, the mode of administration chosen, type of composition, size of a unit dosage, kind of excipients, the age and/or general health of a subject, and other factors well known to those of ordinary skill in the art.

Administration may include a single daily dose or administration of a number of discrete divided doses as may be appropriate. An administration regime may also include administration of one or more of the active agents, or compositions comprising same, as described herein. The period of administration may be variable. It may occur for as long a period is desired.

Administration may include simultaneous administration of suitable agents or compositions or sequential administration of agents or compositions. Where sequential administration of agents is employed, the administration of a second (or third or forth etc) agent or composition need not occur immediately following the administration of the previously administered agent or composition. The method may allow a period of time between administration of a first agent or composition and any subsequently administered agents or compositions.

Exemplary administration regimes are provided herein after within the “Examples” section.

The invention will now be further described with reference to the following non-limiting examples.

### EXAMPLES

### Example 1

Methods

Mice and cell lines. Male C57BL/6 mice, 6-8 weeks old, were obtained from the Animal Resource Unit, Faculty of Medicine and Health Science, University of Auckland, Auckland, New Zealand. The EL-4 thymic lymphoma, which is of C57BL/6(H-2^{b}) origin, was purchased from the American Type Culture Collection (Rockville, Md., USA). It was cultured at 37° C. in DMEM medium (Gibco BRL, Grand Island, N.Y., USA), supplemented with 10% foetal calf serum, 50 U/ml penicillin/streptomycin, 2 mM L-glutamine, 1 mM pyruvate.

Expression plasmids. A cDNA fragment encoding full-length (546 bp) mouse VHL was PCR amplified using IMAGE clone 63956 as a template, and the primers 5′-AGG CGG CGG GGG AGC CCG GTC CTG AGG AGA TGG AGG CTG GGC GGC CGC GGC CGG TGC TGC GCT CG-3′ and 5′-ACT CTC AAG GTG CTC TTG GCT CAG TCG CTG TAT GTC CTT CCG CAC ACT TGG GTA G-3′. The resulting PCR product was used as a template for further amplication with the primers 5′-GGG AAT TCC AAT AAT GCC CCG GAA GGC AGC CAG TCC AGA GGA GGC GGC GGG GGA GCC CGG TCC TG-3′ and 5′-GGT CTA GAT CAA GGC TCC TCT TCC AGG TGC TGA CTC TCA AGG TGC TCT TGG CTC A-3′. The PCR product was subcloned into pCDNA3 (Invitrogen). An antisense pCDNA3 expression vector encoding the 5′-end of HIF-1α (nucleotides 152 to 454; GenBank AF003698) has been described previously (21). All constructs were verified by DNA sequence analysis. A pCDNA3 expression vector encoding the signal peptide and first four kringle regions of mouse plasminogen has been described previously (24).

Gene transfer of expression plasmids in situ and measurement of anti-tumor activity. Purified plasmids were diluted to 1 mg/ml in a solution of 5% glucose in 0.01% Triton X-100, and mixed in a ratio of 1:3 (wt:wt) with DOTAP cationic liposomes (Boehringer Mannheim, Mannheim, Germany), as described previously (22). Tumors were established by injection of 2×10⁵ EL-4 tumor cells into the right flank of mice, and growth determined by measuring two perpendicular diameters. Animals were killed when tumors reached more than 1 cm in diameter, in accord with Animal Ethics Approval (University of Auckland). Once tumors reached either 0.1 cm or 0.4 cm in diameter, they were injected with 100 μl expression plasmid (100 μg). For combinational treatment, reagents were delivered in a timed fashion, where VHL plasmid was injected first, followed by antisense HIF-1α plasmid 48 h later. Empty pCDNA3 vector served as a control reagent. All experiments included 6 mice per group, and each experiment was repeated at least once.

Immunohistochemistry. Tumor cryosections (10 μm) prepared 2 days following injection of plasmids were incubated overnight with either a rabbit polyclonal antibody against a peptide corresponding to N-terminal amino acids 1-181 of VHL (FL-181, Santa Cruz Biotechnology, Inc), a mouse anti-mouse HIF-1α mAb (H1α67, Novus Biologicals, Inc., Littleton, Colo., USA), or a rabbit polyclonal antibody against VEGF (Ab-1, Lab Vision Corporation; CA, USA), or an anti-plasminogen mAb recognising kringles 1-3 (Calbiochem-Novabiochem Corp., CA). Rabbit antibody-stained sections were subsequently incubated for 30 min with appropriate secondary antibodies (VECTASTAIN Universal Quick kit, Vector Laboratories, Burlingame, Calif.), and developed with Sigma FAST DAB (3,3′-diaminobenzidine tetrahydrochloride) and CoCl₂ enhancer tablets (Sigma). Sections were counterstained with Mayer's hematoxylin. The Vector M.O.M. Immunodetection Kit (Vector Laboratories, Inc. Burlingame, Calif.) was used to detect the mouse anti-HIF-1α mAb. The total number of HIF-1α positive cells in 10 randomly selected fields was counted, and the percentage of positive staining cells was calculated (percentage of positive cells=number of positive cells×100/total number of cells). Assessment of vascularity. Methodology to determine tumor vascularity has been described previously (21, 23, 24). Briefly, 10 μm frozen tumor sections prepared 4 days after plasmid injection were immunostained with the anti-CD31 antibody MEC13.3 (Pharmingen, CA). Stained blood vessels were counted in five blindly chosen random fields (0.155 mm²) at 40× magnification, and the mean of the highest three counts was calculated. The concentric circles method (25, 26) was used to assess vascularity, where 5 to 6 tumor sections were analysed for each plasmid-injected tumor.

In situ detection of apoptotic cells. Serial sections of 6 μm thickness were prepared from excised tumors that had been frozen in liquid nitrogen, and stored at −70° C. Terminal deoxynucleotidyl transferase-mediated deoxyuridine triphosphate-digoxigenin nick end labelling (TUNEL) staining of sections was performed using an in situ apoptosis detection kit from Boehringer Mannheim, Germany. Briefly, frozen sections were fixed with 4% paraformaldehyde solution, permeabilized with a solution of 0.1% Triton-X100 and 0.1% sodium citrate, incubated with TUNEL reagent for 60 min at 37° C., and examined by fluorescence microscopy. Adjacent sections were counterstained with haematoxylin and eosin. The total number of apoptotic cells in 10 randomly selected fields was counted. The apoptotic index was calculated as the percentage of positive staining cells, namely AI=number of apoptotic cells×100/total number of nucleated cells.

Western blot analysis. Tumors previously injected with either empty plasmid, or VHL and antisense HIF-1α expression plasmids were excised, minced with scissors and homogenized in protein lysate buffer (50 mmol/L Tris pH 7.4, 100 μmol/L EDTA, 0.25 mol/L sucrose, 1% SDS, 1% NP40, 1 μg/ml leupeptin, 1 μg/ml pepstatin A and 100 μmol/L phenylmethylsulfonyl fluoride) at 4° C. using a motor-driven Virtus homogenizer (Virtus, Gardiner, N.Y.). Tumor lysates from each treatment group were pooled, and debris removed by centrifugation at 10,000×g for 10 min at 4° C. Protein samples (100 μg) were resolved on 10% polyacrylamide SDS gels under reducing conditions, and electrophoretically transferred to nitrocellulose Hybond C extra membranes (Amersham Life Science, Buckingham, England). After blocking the membranes with 5% bovine serum albumin in Tween 20/Tris-buffered saline (TTBS; 20 mmol/L Tris, 137 mmol/L NaCl pH 7.6, containing 0.1% Tween-20), blots were incubated with primary antibodies, and subsequently with horseradish peroxidase-conjugated secondary antibodies. They were developed by enhanced chemiluminescence (Amersham International, Buckingham, England), and exposure to x-ray film. Band density was quantified using Scion Image software (Scion Corporation, Frederick, Md.).

Administration of anti-angiogenic peptides and proteins. VEGF blocking peptides ATWLPPR and a retro-D-isomeric form rpplwta (19) were purchased from Mimotopes, Clayton, Victoria, Australia. Peptides were dissolved in PBS. Mice were randomly assigned to two groups (n=6) and VEGF blocking peptides were injected intratumorally (30 mg/kg body weight) every day for 7 days or subcutaneously (30 mg/kg body weight) every day for two weeks. A mouse endostatin pET11-His6 expression plasmid was constructed with cDNA encoding the 3′-region of mouse collagen XVIII. As reported in the literature, bacterially produced recombinant His-tagged endostatin proved to be largely insoluble. To overcome this problem, we employed methodology described by Huang et al. (27)—the disclosure of which is herein disclosed by way of reference, and produced soluble, active, endostatin for use in the present study. Endostatin was injected intratumorally or subcutaneously at 50 mg/kg of body weight, once a day for 2 weeks.

Statistical analysis. Results were expressed as mean values+standard deviation (SD). A student's t test was used for evaluating statistical significance, where a value less than 0.05 (P<0.05) denotes statistical significance.

Results

VHL synergizes with antisense HIF-1α to completely eradicate large tumors. Combining over-expressed VHL with antisense HIF-1α had surprising effect on large tumors, indicating that the effects of VHL may not be limited to regulating HIF-1α levels and angiogenesis, but may include regulation of the cell cycle, apoptosis, and the extracellular matrix.

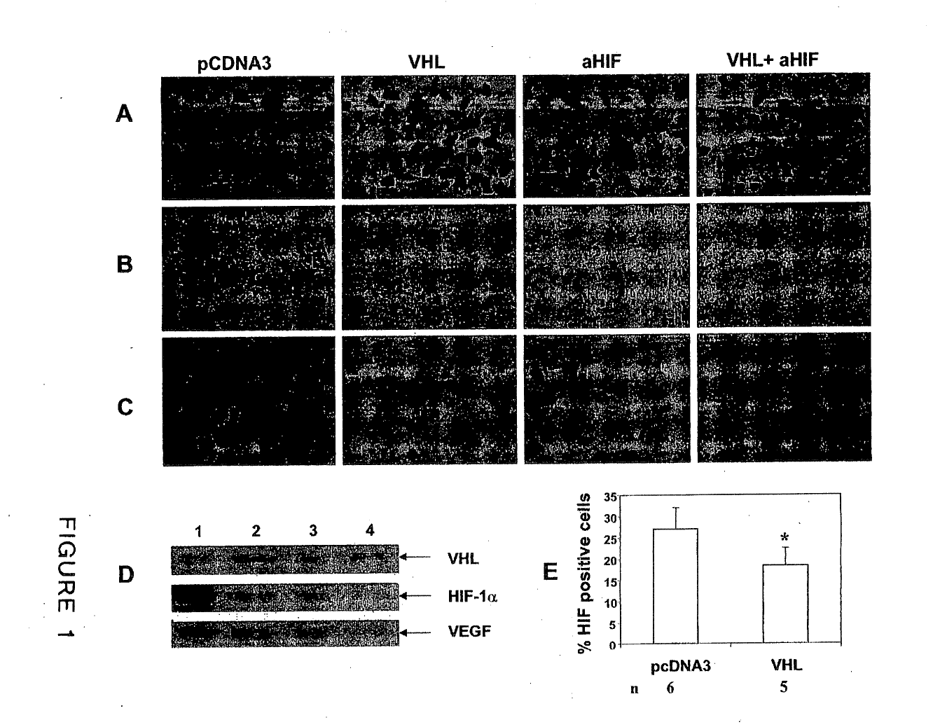

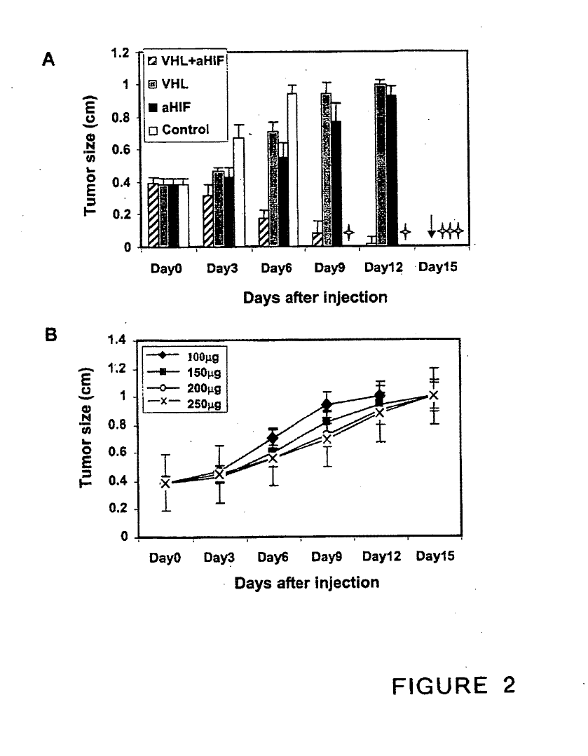

In order to test this, large 0.4 cm diameter tumors were injected with 100 μg of each of the VHL and antisense HIF-1α plasmids. The VHL plasmid was injected first, followed by the HIF-1α antisense plasmid 48 h later, as previous experience has indicated that for whatever reason simultaneous injection of two different plasmids can abrogate their individual effects. Immunohistochemical and Western blot analysis of tumors revealed that VHL was over-expressed in tumors injected with the combination of VHL and antisense HIF-1α plasmid (FIG. 1A). Tumors rapidly and completely regressed within 15 d of plasmid injection (FIG. 2A), and mice remained tumor-free for 3 weeks (data not shown). These same large tumors were refractory to VHL and antisense HIF-1α monotherapies, suggesting that VHL and antisense HIF-1α synergize to eradicate tumors.

Intratumoral injection of VHL plasmids down-regulates the expression of HIF-1α and its effector molecule VEGF. In order to understand the mechanisms responsible, in part, for the anti-tumor activity exhibited by exogenous VHL, we examined tumors that had been injected with VHL plasmid for the levels of HIF-1α, and its effector VEGF. Gene transfer of VHL led to complete downregulation of HIF-1α expression in a proportion (20%) of tumor cells, as revealed by immunohistochemistry (FIGS. 1B and E), and supported by Western blot analysis (FIG. 1D). However, a major proportion of tumor cells appeared to retain some HIF-1α expression (FIGS. 1B and E). In contrast, few cells expressed HIF-1α following antisense HIF-1α therapy administered either alone or combination with exogenous VHL (FIG. 1B). Similarly, both VHL and antisense HIF-1α therapies led to down-regulation of tumoral VEGF expression, where the degree of VEGF loss corresponded to VHL plus antisense HIF-1α therapy>antisense HIF-1α monotherapy>VHL monotherapy (FIGS. 1C and D). VEGF expression was completely lost in tumors injected with a combination of VHL and antisense HIF-1α plasmids.

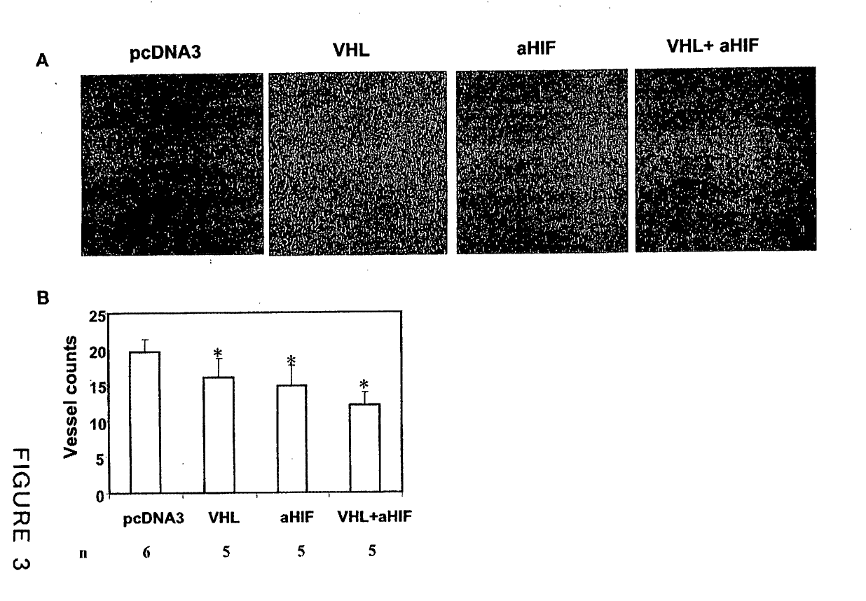

VHL therapy synergizes with anti-sense HIF-1α to reduce tumor blood vessel density, and increase apoptosis. Injection of either VHL or antisense HIF-1α plasmids into tumors inhibited tumor angiogenesis, as evidenced by a statistically significant (p<0.05) reduction in tumor blood vessel density (FIGS. 3A and B), in accord with reductions in the angiogenic factors HIF-1α, and VEGF. The median and 90th centile distances to the nearest CD31-labelled venules from an array of points within tumors treated with VHL or antisense HIF-1α plasmid were significantly (both p<0.05) longer than those for tumors treated with empty vector (Table 1). However, the combination of VHL and antisense HIF-1α was the most effective of all, such that only a few pinpoints of CD31 staining, presumably representing small malformed vessels, were apparent (FIGS. 3A and B). The median and 90th centile distances to the nearest CD31-labelled venules from an array of points within tumors treated with the combination of VHL and antisense HIF-1α plasmid were significantly longer than those for tumors treated with either empty pCDNA3 (P<0.01), VHL (P<0.05), or antisense HIF-1α (P<0.05) plasmid (Table 1).

TABLE 1
Vessel density measured by the concentric circle method

| | Median | | 90th Centile | |
|---|---|---|---|---|
| Plasmid | | P Value | | P Value |
| pcDNA3 | 18.3 ± 5.2 | | 38.3 ± 5.2 | |
| VHL | 25 ± 4.5 | <0.05 | 43 ± 0 | <0.05 |
| aHIF | 27 ± 0 | <0.05 | 45 ± 4.5 | <0.05 |
| VHL + | 29 ± 5.5 | <0.01, 0.05*, | 49 ± 5.5 | <0.01, <0.05*, |
| aHIF | | <0.05** | | <0.05** |

Note:
*Compared with VHL treated tumors;
**Compared with aHIF treated tumors; compared with empty pcDNA3 plasmid where there is no asterisk.

The median and 90th centile distances (±SD) to the nearest CD31-labelled venules from an array of points within tumors injected with either empty pCDNA3 plasmid, VHL, aHIF, or VHL+aHIF plasmids were determined.

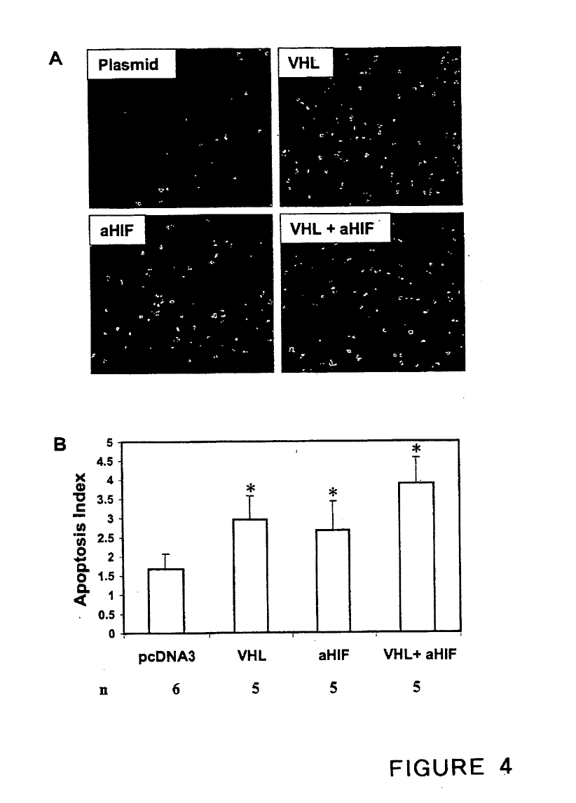

Since tumors were deprived of tumor blood vessels, and survival factors, we examined whether they underwent programmed death as measured by in situ labelling of fragmented DNA using the TUNEL method. A small number of apoptotic cells were detected in tumors injected with empty plasmid (FIG. 4A), whereas tumor apoptosis was almost doubled following injection of either VHL or antisense HIF-1α plasmids (FIG. 4A, and refer to Apoptosis Index in FIG. 4B). Despite the finding that antisense HIF-1α was superior at inhibiting tumor angiogenesis, VHL treatment was more effective at inducing tumor apoptosis, but once again the combination of VHL and antisense HIF-1α was the most effective (FIGS. 4A and B). Thus, the apoptotic index (AI) for tumors injected with VHL, antisense HIF-1α, or a combination of the latter two plasmids was significantly (P<0.001) different from that of tumors treated with empty pCDNA3 vector. The AI for tumors injected with a combination of VHL and antisense HIF-1α plasmids was significantly different from that of tumors injected with either VHL (P<0.05), or antisense HIF-1α (P<0.01) plasmid.

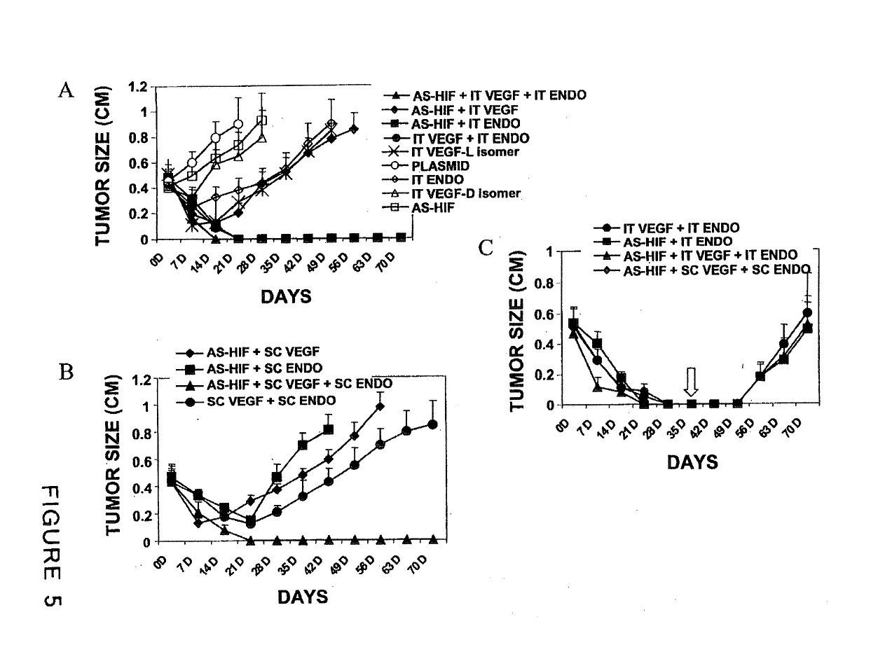

Antisense HIF-1α therapy synergizes with endostatin and VEGF blocking peptide to eradicate large tumors. Endostatin and/or VEGF blocking peptide were administered to mice bearing large tumors (˜0.5 cm in diameter), followed 24 h later by intratumoral injection of antisense HIF-1α plasmids (FIG. 5). Endostatin and/or the normal L-isomeric form of the VEGF blocking peptide were initially administered intratumorally to maintain high local concentrations of these anti-angiogenic agents (FIG. 5A). As monotherapies, both reagents only weakly slowed tumor growth. In contrast, intratumoral injection of the retro-D-isomer of the VEGF blocking peptide had little effect on tumor growth, and hence this isomer was not included in subsequent experiments. As described previously, antisense HIF-1α monotherapy also weakly inhibited tumor growth. In contrast, combined endostatin and antisense HIF-1α therapies caused complete tumor rejection. A combination of all three reagents (endostatin, VEGF peptide, and antisense HIF-1α) was the most effective, causing rapid and complete regression of all tumors.

As a more stringent test of efficacy, endostatin and VEGF blocking peptide were administered subcutaneously, which would be expected to substantially reduce the amount of each agent that reaches the tumor, and more closely represents the route by which these reagents are systemically administered to human patients (FIG. 5B). The triple combination of subcutaneous endostatin, subcutaneous VEGF peptide, and antisense HIF-1α led to complete tumor regression. Mice that were cured by the above treatment regimes were challenged by sc injection of 2×10⁵ parental EL-4 cells into the opposing flank. Tumors grew out in every case indicating that none of the anti-tumor responses matures to an extent that an acquired anti-tumor immunity develops (FIG. 5C).

Discussion

The results given above indicate that use of HIF-1 inhibiting agents, such as antisense HIF-1α therapy is surprisingly able to synergize with systemically administered antiangiogenic agents, endostatin and VEGF blocking peptide, to cause the complete eradication of large tumors, which are refractory to monotherapies. The combination of endostatin and VEGF peptide may be required to directly target the tumour vasculature when systematically administered. Potentially, antisense HIF-1α therapy could synergize with either endostatin or VEGF peptide alone if they were administered systematically in higher amounts. The results lead to the conclusion that those HIF-1 inhibiting agents capable of systemic administration could be combined with antiangiogenic agents capable of systemic administration (eg endostatin plus VEGF blocking peptide) to create a totally systemic therapy. A possible explanation for the synergy, at least in part, is that HIF-1 inhibition by antisense HIF-1α therapy prevents tumors from upregulating hypoxia-inducible factors in response to antiangiogenic (endostatin, and VEGF peptide)-induced hypoxia, thereby preventing tumors from fighting back.

The inventors have demonstrated here that the combination of antisense HIF-1α and VHL therapies leads to an almost complete loss of tumor angiogenesis compared to monotherapies which are not as effective, resulting in the complete regression of large tumors. Unlike conventional anti-angiogenic agents, antisense HIF-1α and VHL therapies inhibit an array of signalling pathways, some unrelated to angiogenesis. While not wishing to be bound by any particular theory, the inventors propose that the combined affect of inhibiting these several pathways is enough to cripple tumor cells, depriving them of key factors required for growth and survival. Whilst, the present study has focussed on intratumoral VHL and anti-sense HIF-1α gene transfer into localized tumors, it will be appreciated that systemic means of delivery, including viral vectors, may provide greater utility for this therapeutic strategy, in particular for patients with systemic disease.

### Example 2

Methods

Mice and Cell Lines.

Male C57BL/6 mice, 6-8 weeks old, were obtained from the Animal Resource Unit, Faculty of Medicine and Health Science, University of Auckland, Auckland, New Zealand. The EL-4 thymic lymphoma, which is of C57BL/6(H-2^{b}) origin, was purchased from the American Type Culture Collection (Rockville, Md., USA). It was cultured at 37° C. in DMEM medium (Gibco BRL, Grand Island, N.Y., USA), supplemented with 10% foetal calf serum, 50 U/ml penicillin/streptomycin, 2 mM L-glutamine, 1 mM pyruvate.

Expression Plasmids.

The pcDNA3 expression vector encoding mouse angiostatin containing 4-kringle of plasminogen and an antisense pcDNA3 expression vector encoding the 5′-end of HIF-1α have been described previously.^{24,21} All constructs were verified by DNA sequence analysis.

Gene Transfer of Expression Plasmids in situ and Measurement of Anti-Tumor Activity.

Purified plasmids were diluted to 1 mg/ml in a solution of 5% glucose in 0.01% Triton X-100, and mixed in a ratio of 1:3 (wt:wt) with DOTAP cationic liposomes (Boehringer Mannheim, Mannheim, Germany), as described previously.^{24,21} Tumors were established by injection of 2×10⁵ EL-4 tumor cells into the right flank of mice, and growth determined by measuring two perpendicular diameters. Animals were killed when tumors reached more than 1 cm in diameter, in accord with Animal Ethics Approval (University of Auckland). Once tumors reached either 0.1 cm or 0.4 cm in diameter, they were injected with 100 μl expression plasmid (100 μg). For combinational treatment, reagents were delivered in a timed fashion, where angiostatin plasmid was injected first, followed by antisense HIF-1α plasmid 24 h later. Empty pcDNA3 vector served as a control reagent. All experiments included 6 mice per group, and each experiment was repeated at least once.

Immunohistochemistry.

Tumor cryosections underwent overnight incubation with either an anti-plasminogen mAb recognizing kringles 1-3 (Calbiochem-Novabiochem Corp., CA), a rabbit polyclonal antibody against VEGF (Ab-1, Lab Vision Corp., CA), or an anti-mouse HIF-1α mAb (H1α67, Novus Biologicals, Inc., Littleton, Colo., USA). The sections were then subsequently incubated for 30 min with appropriate secondary antibodies (VECTASTAIN Universal Quick kit, Vector Laboratories, Burlingame, Calif.), and developed with Sigma FAST DAB (3,3′-diaminobenzidine tetrahydrochloride) and CoCl₂ enhancer tablets (Sigma). Sections were counterstained with Mayer's hematoxylin.

Western Blot Analysis.

Tumors previously injected with expression plasmids were excised at pre-scheduled time, and homogenized in protein lysate buffer. Protein samples (100 μg) were resolved by SDS-PAGE, and electrophoretically transferred to nitrocellulose Hybond C extra membranes. The membranes were incubated with primary antibodies, and subsequently with horseradish peroxidase-conjugated secondary antibodies. They were developed by enhanced chemiluminescence (Amersham International, Buckingham, England), and exposure to x-ray film. Band density was quantified using Sigma ScanPro software.

Assessment of Vascularity.

Ten-μm frozen tumor sections prepared 4 days after plasmid injection were immunostained with the anti-CD31 antibody MEC13.3 (Pharmingen, CA). Stained blood vessels were counted in five blindly chosen random fields (0.155 mm²) at 40× magnification, and the mean of the highest three counts was calculated. The concentric circles method was used to assess vascularity, where 5 to 6 tumor sections were analysed for each plasmid-injected tumor.

In Situ Detection of Apoptotic Cells.

Frozen sections of 6 μm thickness were prepared from excised tumors. After fixation, and permeablization, the sections were incubated with terminal deoxynucleotidyl transferase-mediated deoxyuridine triphosphate-digoxigenin nick end labelling (TUNEL) staining reagent for 60 min at 37° C., and examined by fluorescence microscopy. Adjacent sections were counterstained with haematoxylin and eosin. The total number of apoptotic cells in 10 randomly selected fields was counted. The apoptotic index was calculated as the percentage of positive staining cells, namely AI=number of apoptotic cells×100/total number of nucleated cells.

Statistical Analysis.

Results were expressed as mean values+standard deviation (SD). A student's t test was used for evaluating statistical significance, where a value less than 0.05 (P<0.05) denotes statistical significance.

Results

Blocking Induction of Hypoxic Inducible Pathways by Inhibiting HIF-1α Circumvents Acquired Resistance to Anti-Angiogenic Drugs

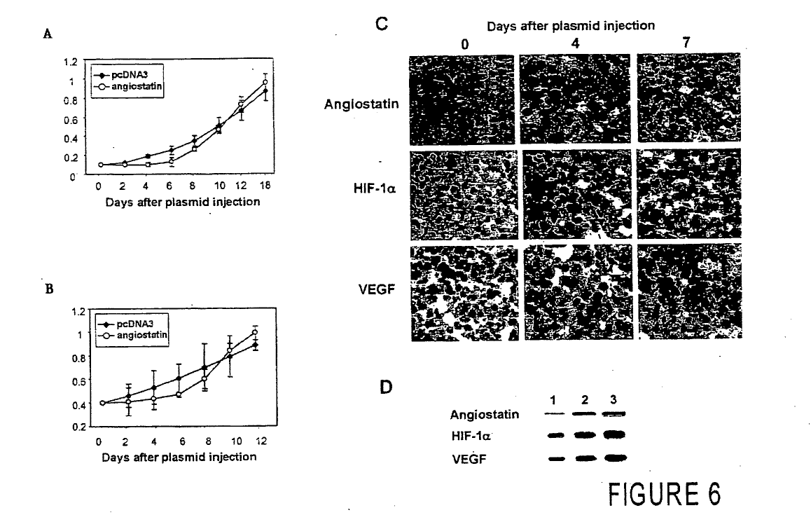

Tumors treated with angiostatin display drug resistance. EL-4 tumors of 0.1 cm (FIG. 6A) and 0.4 cm (FIG. 6B) in diameter were established in the flanks of C57BL/6 mice, and injected with a DNA/liposome transfection vehicle containing either 100 μg of angiostatin plasmid DNA or 100 μg of empty vector control. Tumors grew rapidly in the control group, reaching 1 cm in size 15 to 18 d following gene transfer. In contrast, the growth of tumors treated with angiostatin plasmid was suppressed for 6 d after angiostatin gene transfer, but then tumors grew rapidly with growth out-stripping even the controls. Immunohistochemical analysis of tumor sections prepared 4 and 7 d following gene transfer, revealed angiostatin gene therapy resulted in stable overexpression of angiostatin in situ for at least one week (FIG. 6C). Surprisingly, expression of HIF-1α and its effector VEGF was upregulated within 4 days of angiostatin treatment, and was further increased by day 7 (FIG. 6C). These results were confirmed by Western blot analysis of tumor homogenates (FIG. 6D). The results indicate that angiostatin treatment upregulates the expression of HIF-1α and VEGF, leading to drug-resistance, and accelerated tumor growth.

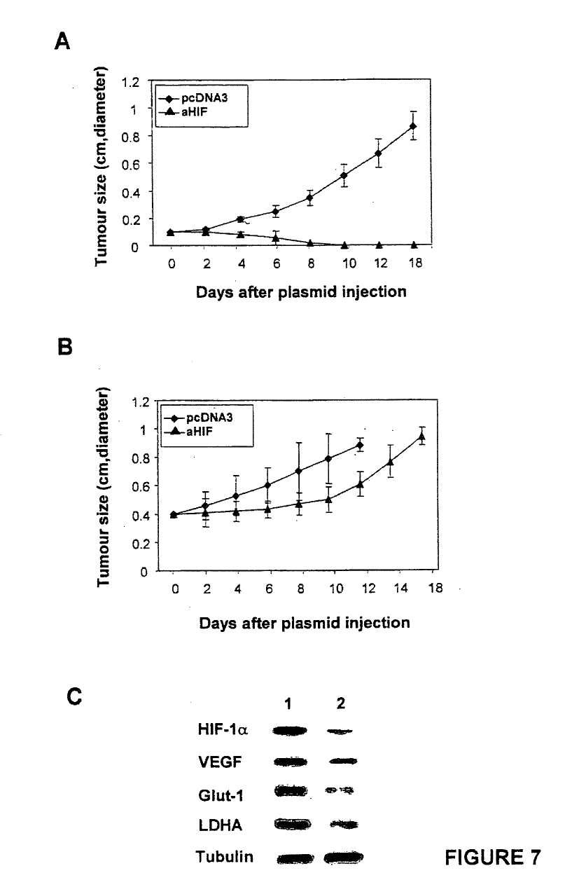

Tumors treated with antisense HIF-1α do not develop drug resistance. EL-4 tumors of 0.1 cm (FIG. 7A) and 0.4 cm (FIG. 7B) in diameter were established in C57BL/6 mice, and injected with DNA/liposome transfection vehicle containing either 100 μg of antisense HIF-1α expression plasmid or 100 μg of empty vector control. Tumors grew rapidly in the control groups, whereas small 0.1 cm tumors treated with the antisense HIF-1α plasmid completely and rapidly regressed within two weeks of gene transfer (FIG. 7A), as described previously.²¹ Large 0.4 cm tumors were significantly (P<0.01) slowed in their growth by antisense HIF-1α therapy, but none of the tumors completely regressed. The failure of antisense HIF-1 α therapy against large tumors was not the result of an inadequate dosage of plasmid, as increasing the dosage to 250 μg did not significantly improve the inhibition of tumor growth (data not shown), in accordance with a previous study.²¹ Western blot analysis of tumor homogenates, prepared 2 d following gene transfer, revealed antisense therapy resulted in almost complete loss of expression of HIF-1α and its downstream effectors VEGF, and Glut1 and LDHA (FIG. 7C). Thus, blockade of HIF-1 in EL-4 tumors does not lead to the upregulation of angiogenic factors such as VEGF as seen with angiostatin therapy.

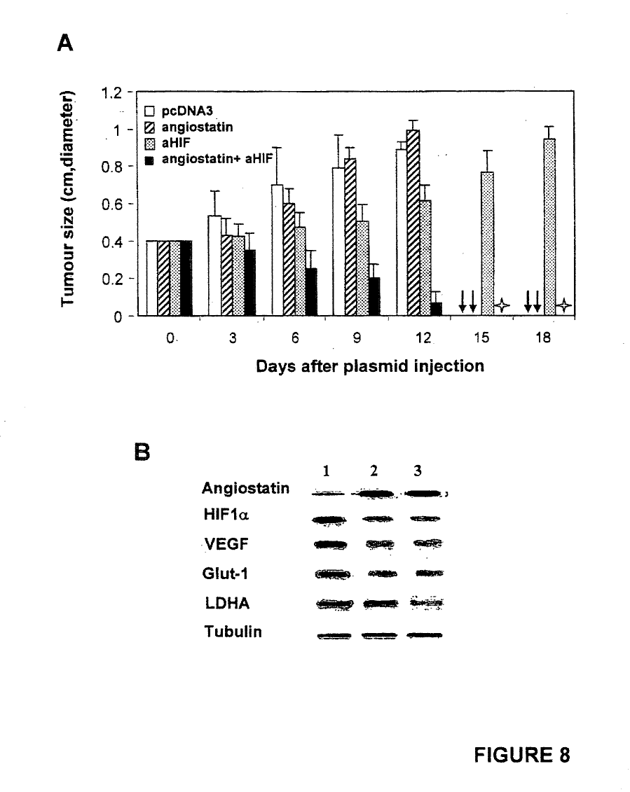

Antisense HIF-1α synergizes with angiostatin to eradicate large tumors. EL-4 tumors of 0.4 cm in diameter were treated with a combination of 100 μg angiostatin plasmid, and 100 μg of antisense HIF-1α expression plasmid, where angiostatin plasmid was injected first followed 24 h later by antisense HIF-1α. Tumors injected with either empty vector, angiostatin plasmid, or antisense HIF-1α, served as controls. The control plasmids in the combinational experiment were also injected twice in a similarly timed fashion. The combination of angiostatin and antisense HIF-1α plasmids led to complete tumor regression within two weeks, and mice remained tumor-free for 2 months (FIG. 8A). In contrast, none of the tumors in the three control groups of mice regressed completely. However, tumors treated with antisense HIF-1α were slowed in their growth compared to tumors treated with angiostatin or empty vector (FIG. 8A). Western blot analysis of tumors prepared 4 and 10 days following gene transfer revealed that combinational gene therapy prevented the upregulation of HIF-1α and VEGF in response to angiostatin. Rather, expression of HIF-1α and its downstream effectors VEGF, Glut1 and LDHA was greatly reduced four days following plasmid injection, and suppressed until at least day 10 (FIG. 8B).

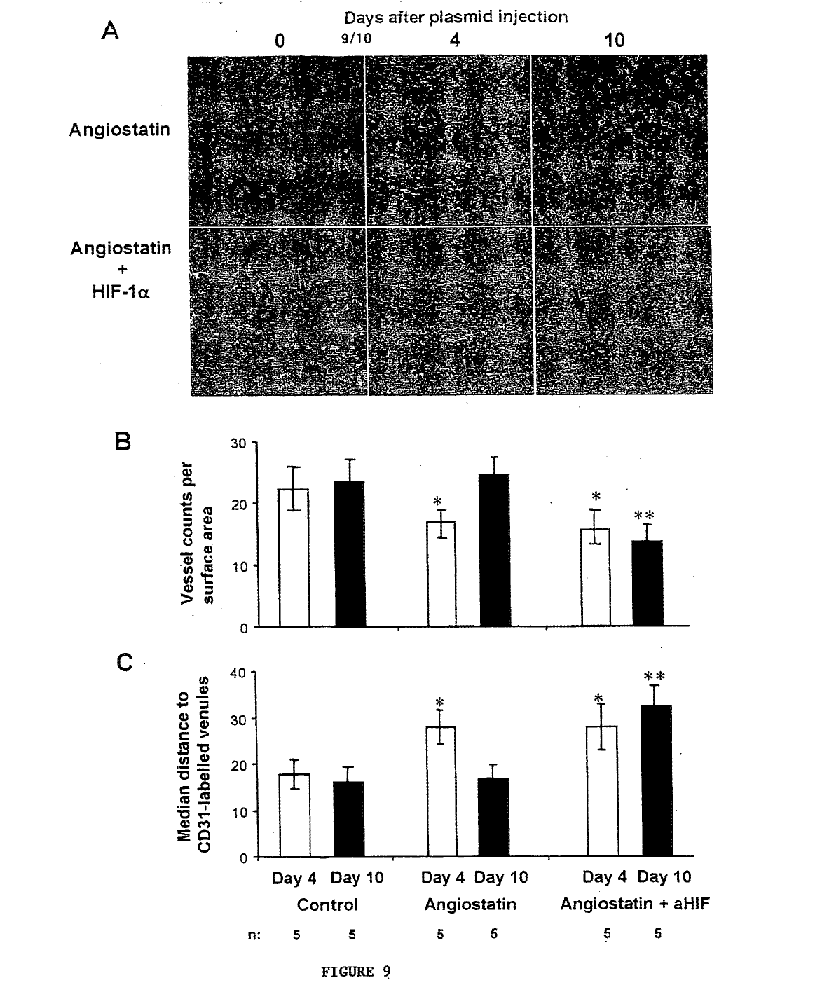

Antisense HIF-1α therapy synergizes with angiostatin to inhibit tumor angiogenesis. The inventors sought to determine whether accelerated tumor growth 10 days following angiostatin treatment was due to increased tumor angiogenesis, given that a single injection of angiostatin plasmid led to upregulation of HIF-1α and VEGF. Tumors (0.4 cm in diameter) that had been injected with 100 μg of angiostatin plasmid were removed on days 4 and 10, sectioned, and stained with an anti-CD31 mAb to visualize tumor blood vessels. Angiostatin gene therapy resulted in a statistically significant (P<0.01) reduction in tumor vascularity by day 4, in accord with a previous study (21), however by day 10 blood vessel density had increased to be slightly greater than that of control tumors treated with empty vector (FIGS. 9A and B). The median distance to the nearest anti-CD31 mAb-labeled vessels from an array of points within the tumors treated with angiostatin was significantly longer than that for tumors treated with empty vector on day 4, but had shortened by day 10 (FIG. 9C). In contrast, tumors injected with the combination of angiostatin plasmid, and antisense HIF-1α had significantly (P<0.01) reduced blood vessel density on day 4, and even less on day 10 (P<0.001), compared to tumors injected with empty vector. The median distance to the nearest anti-CD31-labeled vessels from an array of points within tumors treated with combinational therapy was significantly lengthened by days 4 (P<0.01) and 10 (P<0.001), compared to tumors injected with empty vector (FIG. 9C).

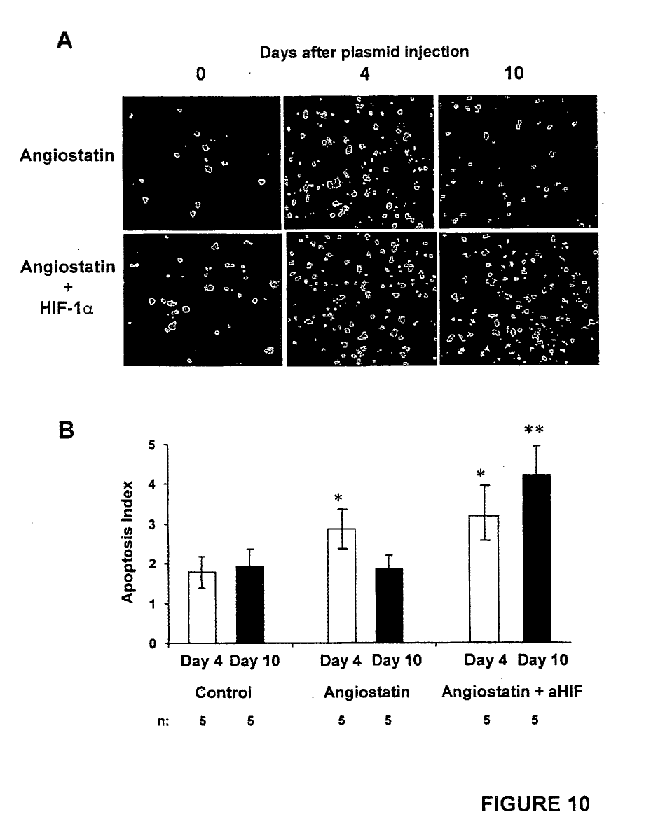

Antisense HIF-1α therapy synergizes with angiostatin to induce tumor cell apoptosis. The inventors next examined whether tumors underwent programmed cell death as measured by the TUNEL method, given that they were deprived of either tumor blood vessels or survival factors after therapy. Small numbers of apoptotic cells were detected in tumors injected with empty plasmid, whereas tumor apoptosis was almost doubled following injection of angiostatin on day 4. However, by day 10 tumor apoptosis had declined to levels seen in tumors injected with empty plasmid (FIGS. 10A and B). In contrast, tumor apoptosis increased in response to combination therapy by day 4 (P<0.01), and was further increased by day 10 (P<0.001).

Discussion

Angiogenesis inhibitors have been classified into two groups, namely ‘direct’ and ‘indirect’ inhibitors.²⁹ Direct inhibitors such as angiostatin prevent vascular endothelial cells from proliferating, or migrating to pro-angiogenic proteins, including VEGF. It has been argued that direct angiogenesis inhibitors are the least likely to induce acquired drug resistance, because they target genetically stable endothelial cells rather than unstable mutating tumor cells.^{16, 29} Thus, tumors treated with direct-acting endostatin therapy did not develop drug resistance in mice.¹⁶ Nevertheless, the inventors have demonstrated here that EL-4 tumors rapidly become resistant to angiostatin, which initially suppresses tumor growth for 6 days. Tumors are soon faced with increasing hypoxia in response to angiostatin treatment. They respond within one week of therapy by upregulating the expression of HIF-1α, and its effector VEGF, leading to increased tumor vascularity, decreased tumor apoptosis, and accelerated tumor growth, despite the fact that high levels of exogenous angiostatin are maintained throughout. Thus, drug resistance to direct anti-angiogenic therapy lies not with the endothelial cells, but with the tumor cells that remain capable of upregulating hypoxia-inducible pathways, producing factors that either directly or indirectly out-compete angiostatin.

Indirect angiogenesis inhibitors are classified as preventing the expression of or blocking the activity of a tumor protein, such as VEGF, that activates angiogenesis, or blocking the expression of its receptor on endothelial cells.²⁹ Angiostatin can be viewed as both a direct and indirect inhibitor, as it has several effects on endothelial cells. As a direct inhibitor it inhibits endothelial proliferation by binding to the α/β-subunits of ATP synthase,³⁰ blocks αVβ3 function,³¹ inhibits the activation of plasminogen in the extracellular matrix,³² induces apoptotic cell death,³³ subverts adhesion plaque formation and thereby inhibits migration and tube formation stimulated by angiomotin.^{34,35} As an indirect inhibitor it has also been shown to down-regulate VEGF expression.^{36,37} It is argued that indirect inhibitors are prone to cause resistance, as tumors that begin to express proangiogenic factors not affected by a particular indirect angiogenesis inhibitor will start to outgrow.²⁹ The results here suggest that tumors may in addition become drug-resistant by upregulating the expression of targets of indirect angiogenesis inhibitors, as evidenced by the upregulation of VEGF in response to angiostatin. Variants of A431 squamous cell carcinoma tumor cells are another example of this resistance phenomenon. They display acquired resistance to anti-EGFR antibodies, which block the production of several proangiogenic growth factors, including VEGF, interleukin-8, and basic fibroblast growth factor.³⁸ In this case, resistant A431 variants emerge in vivo, at least in part, by mechanisms involving the selection of tumor cell subpopulations with increased angiogenic potential.

The problem with current anti-angiogenic cancer therapies is that they target angiogenic factors downstream of the HIF-1, the master regulator of oxygen homeostasis. This enables tumor cells to sense they are deprived of oxygen, and respond accordingly by upregulating their pro-angiogenic arsenal. In contrast, as shown here EL-4 tumors could not circumvent antisense HIF-1α treatment by upregulating proangiogenic factors such as VEGF, or other tumor survival factors. This finding suggests EL-4 tumors cells do not express other HIFα subunits, which could otherwise upregulate hypoxia-inducible pathways, and antagonize treatment. To remain effective most of the angiogenesis inhibitors undergoing human trial must be administered on a dose-schedule that maintains a constant concentration in the circulation capable of out-competing tumor-expressed angiogenic factors. Hence, repeat injections of angiostatin expression plasmid achieved better results than a single injection, and the degree of tumor growth inhibition appears to be directly proportional to the levels of expression of the angiostatin transgene.³⁹ The anti-angiogenic drugs in trials cause tumor regression in but a few patients, and most patients experience only tumor stabilization.⁴⁰ Tumor regression by anti-angiogenic therapy is slow, and can take more than 1 year.^{41,42} The present results suggest that if anti-angiogenic treatment is suspended or ineffectual then a possible outcome is accelerated tumor growth. It has been suggested that a combination of two or more angiogenesis inhibitors may prevent drug resistance, as evidenced by the fact that mice injected with retrovirally transformed tumor cells overexpressing angiostatin and endostatin,⁴³ have increased survival compared to those receiving tumors singly transformed with either angiostatin or endostatin. The inventors have surprisingly found here that greater efficacy could be achieved by inhibiting HIF-1 to prevent tumors from sensing hypoxia, and thereby circumvent acquired resistance to angiostatin. As described, the timed injection of a combination of angiostatin and antisense HIF-1α plasmids into large tumors resistant to the respective monotherapies led to prolonged suppression of tumor angiogenesis, enhanced tumor cell apoptosis, and complete tumor regression. Increased tumor apoptosis is in accord with several studies that indicate that angiogenesis inhibitors can induce tumor-cell apoptosis by decreasing levels of an array of endothelial-cell-derived paracrine factors that promote tumor cell survival.^{44,45} Combination therapy did not succumb to acquired drug resistance, but rather durably suppressed the expression of HIF-1α and VEGF, as well as the tumor survival factors Glut-1 and LDHA. Thus, anti-sense HIF-1α therapy prevented acquired tumor resistance to angiostatin. In turn, angiostatin augmented antisense HIF-1α therapy, as the latter alone could only slow the growth of large tumors. Angiostatin may antagonize the function of other pro-angiogenic factors, such as hepatocyte growth factor, whose expression is not necessarily hypoxia-dependent,⁴⁶ but this point was not examined further here.

Some tumors express increased levels of HIF-1 due to acquired mutations in regulatory genes, rather than as a response to hypoxia. Such mutations antagonize anti-angiogenic therapy by increasing the total angiogenic profile of a tumor. For instance, tumors in which the p53 tumor suppressor gene has been inactivated (about 50% of human cancers) are much less responsive to angiogenesis inhibitors than comparable tumors in which the gene is still functional.⁴⁷ P53 normally suppresses tumor angiogenesis by upregulating TSP1,⁴⁸ inducing the degradation of HIF-1α⁴⁹ Mutations in p53 lead to enhanced levels of HIF-1α, and augmented HIF-1-dependent transcriptional activation of VEGF. Thus, blockade of HIF-1 could also prevent neovascularization due to the outgrowth of tumor cell variants that express increased levels of angiogenic factors due to the loss of function of p53.

In summary, the data provided herein above suggest that anti-cancer treatments directed against the tumor vasculature should be accompanied by therapies that target HIF-1α subunits expressed by tumors, in order to prevent tumors from developing resistance to drug-induced hypoxia and starvation. Such therapies could also prevent the selective outgrowth of tumor cells with a strong angiogenic profile arising from gene mutations that stabilize HIF-1α subunits. Coadministration of drugs that directly or indirectly target HIF-1, particularly antisense HIF-1α, could render the large number of anti-angiogenic drugs currently undergoing human clinical trials far more effective. Currently, the rationale underlying long-term (several years) administration of angiogenesis inhibitors is to achieve and maintain “stable disease”. In contrast, the combination strategies described here could potentially achieve complete tumor regression within a relatively short time-frame.

Careful consideration has to be given to choosing therapeutic anti-angiogenic reagents that are most likely to synergize with one another, if drug resistance is to be prevented, and tumors eradicated. The results herein reveal that administration of a combination of anti-angiogenic factors that simultaneously act both on tumor and on the tumor endothelium may be required to completely block the angiogenic cascade and tumor growth.

It has been demonstrated that combining HIF-1 inhibition by antisense HIF-1α therapy with VHL therapy leads to a further loss of HIF-1α, and VEGF, and tumor angiogenesis compared to monotherapies, resulting in the complete eradication of large tumors. The resulting tumour eradication is unexpected as neither active individually has this effect. It is thought that the combined effect is enough to cripple tumor cells, and potentially expose them to the innate immune system which senses danger signals from damaged cells. Again, the potential for systemic treatment by combination of HIF-1 inhibiting agents with factors that increase VHL in tumors or that mimic VHL function, could be a major advance in cancer treatment.

In addition it has been demonstrated that combining HIF-1 inhibition by antisense HIF-1α therapy with targeting VEGF function, or with angiostatin or endostatin therapies, one can achieve complete eradication of tumors. These results are again unexpected having regard to the fact that none of the active agents alone produce such results.

The results herein also indicate that targeting tumors by inhibiting HIF and preventing the upregulation by tumors of hypoxia-inducible factors, coupled with anti-angiogenic agents that target the growth, and/or survival of tumor endothelial cells is a very effective approach.

Following the applicant's surprising determination of the effect of the combinations described herein, effective dose rates for larger animals, eg humans, would simply be a matter of trial and error, within the abilities of the skilled person to determine. The issue of effect against large tumors (or small tumors) is thus equally a matter of trial and error.

The option of administering action agents used in the combination treatment subcutaneously, thus providing a systemic treatment approach, is very advantageous in terms of patient comfort and safety. The option of a systemic, or partially systemic, treatment approach provides a major advance in cancer treatment practice.

While in the foregoing description there has been made reference to specific components or integers of the invention having known equivalents then such equivalents are herein incorporated as if individually set forth.

The invention has been described herein with reference to certain preferred embodiments. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country of the world.

Throughout this specification, and the claims which follow, unless the context requires otherwise, the words “comprise”, “comprising” and the like, are to be construed in an inclusive sense as opposed to an exclusive sense, that is to say, in the sense of “including, but not limited to”.

### REFERENCES

1. Blancher, C., and Harris, A. L. 1998. The molecular basis of the hypoxia response pathway: Tumor hypoxia as a therapy target. Cancer Metastasis Rev. 17:187-194, 1998.
2. Wang, G. L., and Semenza, G. L. 1993. General involvement of hypoxia-inducible factor-1 in transcriptional response to hypoxia. Proc. Natl. Acad. Sci. USA 90: 4304-4308.
3. Wang, G. L., Jiang, B-H., Rue, E. A, and Semenza, G. L. 1995. Hypoxia-inducible factor 1 is a basic-helix-loop-helix-PAS heterodimer regulated by cellular O₂ tension. Proc. Natl. Acad. Sci. USA 92:5510-5514.
4. Kondo, K., and Kaelin, W. G. 2001. The von Hippel-Landau tumor suppressor gene. Expt. Cell. Res. 264:117-125.
5. McKusick, V. A. 1992. Mendelian Inheritance in Man. Baltimore and London: Johns Hopkins University Press.
6. Krek, W. 2000. VHL takes HIF's breath away. Nature Cell Biol. 2:E1-E3.
7. Ohh, M., Park, C. W., Ivan, M., Hoffman, M. A., Kim, T. Y., Huang, L. E,. Pavletich, N., Chau, V., and Kaelin, W. G. 2000. Ubiquitination of hypoxia-inducible factor requires direct binding to the beta-domain of the von Hippel-Lindau protein. Nat. Cell. Biol. 2:423-427.
8. Yu, F., White, S. B., Zhao, Q., and Lee, F. S. 2001. HIF-1α binding to VHL is regulated by stimulus-sensitive proline hydroxylation. Proc. Natl. Acad. Sci. USA 98:9630-9635.
9. Jiang B H et al. 1996. Dimerization, DNA binding, and transactivation properties of hypoxia-inducible factor 1. J. Biol. Chem. 271:17771-17778.
10. Ratcliffe, P. J., O'Rourke, J. F., and Maxwell P H, Pugh C W. 1998. Oxygen sensing, hypoxia-inducible factor-1 and the regulation of mammalian gene expression. J. Exp. Biol. 201:1153-1162.
11. Semenza, G. L., Jiang, B. H., Leung, S. W., Passantino, R., Concordet, J. P., Maire, P., and Giallongo, A. 1996. Hypoxia response elements in the aldolase A, enolase 1, and lactate dehydrogenase A gene promoters contain essential binding sites for hypoxia-inducible factor 1. J. Biol. Chem. 271:32529-32537.
12. Dachs, G. U., and Stratford, I. J. The molecular response of mammalian cells to hypoxia and the potential for exploitation in cancer therapy. Br. J. Cancer 1996; 74:S126-132.
13. Iliopoulos, O., Kibel, A., Gray, S., and Kaelin, W. G. 1995. Tumor suppression by the human von Hippel-Lindau gene product. Nature Med. 1:822-826.
14. O'Reilly, M. S., Boehm, T., Shing, Y., Fukai, N., Vasios, G., Lane, W. S., Flynn, E., Birkhead, J. R., Olsen, B. R. and Folkman, J. Endostatin: An endogenous inhibitor of angiogenesis and tumor growth. Cell 1997, 88: 277-285.
15. Muragaki, Y., Timmons, S., Griffith, C. M., Oh, S. P., Fadel, B., Quertermous, T. and Olsen, B. R. Mouse col18a1 is expressed in a tissue-specific manner as three alternative variants and is localized in basement membrane zones. Proc. Natl. Acad. Sci. USA 1995, 92, 8763-8767.
16. Boehm, T., Folkman, J., Browder, T. and O'Reilly, M. S. Antiangiogenic therapy of experimental cancer does not induce acquired drug resistance. Nature 1997, 390: 404-407.
17. Rehn, M., Veikkola, T., Kukk-Valdre, E., Nakamura, H., Ilmonen, M., Lombardo, C. R., Pihlajaniemi, T., Alitalo, K., and Vuori, K. Interaction of endostatin with integrins implicated in angiogenesis. Proc. Natl. Acad. Sci. USA 2001, 98: 1024-1029.
18. Furumatsu, T., Yamaguchi, N., Nishida, K., Kawai, A., Kunisada, T., Namba, M., Inoue, H. and Ninomiya, Y. Endostatin inhibits adhesion of endothelial cells to collagen I via alpha(2)beta(1) integrin, a possible cause of prevention of chondrosarcoma growth. J. Biochem. 2002, 131: 619-626.
19. Binetruy-Tornaire, R., Demangel, C., Malavaud, B., Vassy, R., Rouyre, S., Kraemer, M., Plouet, J., Derbin, C., Perret, G., and Mazie, J. C. Identification of a peptide blocking vascular endothelial growth factor (VEGF)-mediated angiogenesis. EMBO J. 2000, 19:1525-1533.
20. Jones, M. K., Szabo, I. L., Kawanaka, H., Husain, S. S., and Tarnawski, A. S. Von Hippel-Lindau tumor suppressor and HIF-1 alpha: new targets of NSAID inhibition of hypoxia-induced angiogenesis. FASEB J. express 2001, 10.1096/fj.01-0589fje.
21. Sun, X., Kanwar, J. R., Leung, E., Lehnert, K., Wang, D., and Krissansen, G. W. 2001. Gene transfer of antisense hypoxia inducible factor-1α enhances the therapeutic efficacy of cancer immunotherapy. Gene Therapy 8:638-645.
22. Kanwar, J. R., Berg, R. W., Lehnert, K., and Krissansen G. W. 1999. Taking lessons from dendritic cells: multiple xenogeneic ligands for leukocyte integrins have the potential to stimulate anti-tumor immunity. Gene Therapy 6:1835-1844.
23. Heather, E. R., Jessica, L., and Randall, S. J. 1998. HIF-1α is required for solid tumor formation and embryonic vascularization. _i EMBO J. 17:3005-3015.
24. Sun, X., Kanwar, J. R., Leung, E., Lehnert, K., Wang, D., and Krissansen, G. W. 2001. Angiostatin enhances B7.1-mediated cancer immunotherapy independently of effects on vascular endothelial growth factor expression. Cancer Gene Ther. 8:719-727.
25. Kayar, S. R., Archer, P. G., Lechher, A. J., and Banchero, N. 1982. Evaluation of the concentric-circles method for estimating capillary-tissue diffusion distances. Microvascular Res. 24:342-353.
26. Maxwell, P. H., Dachs, G. U., Gleadle, J. M., Nicholls, L. G., Harris, A. L., Stratford, I. J., Hankinson, O., Pugh, C. W., and Ratcliffe, P. J. 1997. Hypoxia-inducible factor-1 modulates gene expression in solid tumors and influences both angiogenesis and tumor growth. Proc. Natl. Acad. Sci. USA 94:8104-8109.
27. Huang, X., Wong, M. K. K., Zhao, Q., Zhu, Z., Wang, K. Z. Q., Huang, N., Ye, C., Gorelik, E., and Li, M. Soluble recombinant endostatin purified from Escherichia coli: Antiangiogenic activity and antitumor effect. Cancer Res. 2001, 61: 478-481.
28. O'Reilly, M. S., Holmgren, L., Shing, Y., et al. 1994. Angiostatin: a novel angiogenesis inhibitor that mediates the suppression of metastases by a Lewis lung carcinoma. Cell 79: 315-328.
29. Kerbel, R. S., and Folkman, J. 2002. Clinical translation of angiogenesis inhibitors. Nature Rev. Cancer 2: 727-739.
30. Moser, T. L., Stack, M. S., Asplin, I., et al. 1999. Angiostatin binds ATP synthase on the surface of human endothelial cells. Proc. Natl. Acad. Sci. USA 96:2811-2816.
31. Tarui, T., Miles, L. A., and Takada, Y. 2001. Specific interaction of angiostatin with integrin αVβ3 in endothelial cells. J. Biol. Chem. 276: 39562-39568.
32. Stack, M. S., Gately, S., Bafetti, L. M., Enghild, J. J., and Soff, G. A. 1999. Angiostatin inhibits endothelial and melanoma cellular invasion by blocking matrix-enhanced plasminogen activation. Biochem. J. 340: 77-84.
33. Holmgren, L., O'Reilly, M. S., and Folkman, J. 1995. Dormancy of micrometastases-balanced proliferation and apoptosis in the presence of angiogenesis suppression. Nature Med. 1: 149-153.
34. Claesson-Welsh, L., Welsh, M., Ito, N., et al. 1998. Angiostatin induces endothelial cell apoptosis and activation of focal adhesion kinase independently of the integrin-binding motif RGD. Proc. Natl. Acad. Sci. USA. 95: 5579-5583.
35. Troyanovsky, B., Levchenko, T., Mansson, G., Matvijenko, O., Holmgren, L. 2001. Angiomotin: an angiostatin binding protein that regulates endothelial cell migration and tube formation. J. Cell Biol. 152: 1247-1254.
36. Kirsch, M., Strasser, J., Allender, R., et al. 1998. Angiostatin suppresses malignant glioma growth in vivo. Cancer Res. 58: 4654-4659.
37. Joe, Y. A., Hong, Y. K., Chung, D. S., et al. 1999. Inhibition of human malignant glioma growth in vivo by human recombinant plasminogen kringles 1-3. Int. J. Cancer 82: 694-699.
38. Viloria-Petit, A., Crombet, T., Jothy, S., Hicklin, D., Bohlen, P., Schlaeppi, J. M., Rak, J., and Kerbel, R S. 2001. Acquired resistance to the antitumor effect of epidermal growth factor receptor-blocking antibodies in vivo: A role for altered tumor angiogenesis. Cancer Res. 61: 5090-5101.
39. Ambs, S., Dennis, S., Fairman, J., Wright, M., Papkoff, J. 1999. Inhibition of tumor growth correlates with the expression level of a human angiostatin transgene in transfected B16F10 melanoma cells. Cancer Res. 59: 5773-5777.
40. Novak, K. 2002. Angiogenesis inhibitors revised and revived at AACR. American Association for Cancer Research. Nature Med. 8: 427.
41. Kaban, L. B. et al. 1999. Anti-angiogenic therapy of a recurrent giant cell tumor of the mandible with interferon alfa-2α Pediatrics 103: 1145-1149.
42. Marler, J. J. et al. 2002. Successful anti-angiogenic therapy of giant cell angioblastoma with interferon α2β: report of two cases. Pediatrics 109: 1-5.
43. Scappaticci, F. A. et al. 2001. Combination angiostatin and endostatin gene transfer induces synergistic activity in vitro and antitumor efficacy in leukemia and solid tumors in mice. Mol. Ther. 3: 186-196.
44. Rak, J. W., St Croix, B. D., and Kerbel, R. S. 1995. Consequences of angiogenesis for tumor progression, metastasis and cancer therapy. Anti-Cancer Drugs 6: 3-18.
45. Dixelius, J. et al. 2000. Endostatin-induced tyrosine kinase signaling through the Shb adaptor protein regulates endothelial cell apoptosis. Blood 95: 3403-3411.
46. Wajih, N., and Sane, D. C. 2003. Angiostatin selectively inhibits signaling by hepatocyte growth factor in endothelial and smooth muscle cells. Blood 101: 1857-1863.
47. Yu, J. L., Rak, J. W., Coomber, B. L., Hicklin, D. J., and Kerbel, R. S. 20002. Effect of p53 status on tumor response to antiangiogenic therapy. Science 295: 1526-1528.
48. Dameron, K. M., Volpert, O. V., Tainsky, M. A., and Bouck, N. 1994. Control of angiogenesis in fibroblasts by p53 regulation of thrombospondin-1. Science 265: 1582-1584.
49. Ravi, R., Mookerjee, B., Bhujwalla, Z. M., Sutter, C. H., Artemov, D., Zeng, Q., Dillehay, L. E., Madan, A., Semenza, G. L., and Bedil, A. et al. 2000. Regulation of tumor angiogenesis by p53-induced degradation of hypoxia-inducible factor-1α Genes Dev. 14: 34-44.
50. Browder, T. et al. 2000. Anti-angiogenic scheduling of chemotherapy improves efficacy against experimental drug-resistant cancer. Cancer Res. 60: 1878-1886.
51. Ambrosini, G., Adida, C., and Altieri, D. C. 1997. A novel anti-apoptosis gene. Survivin expression in cancer and lymphoma. Nat. Med. 3: 917-921.
52. Webb, A., Cunningham, D., Cotter, F., Clarke, P. A., di Stefano, F., Ross, P., et al. 1997. BCL-2 antisense therapy in patients with non-Hodgkin lymphoma. Lancet 349:1137-1141.
53. Miayake, H., Tolcher, A., and Gleave, M. E. 2000. Chemosensitization and delayed androgen-independent recurrence of prostate cancer with the use of antisense Bcl-2 oligodeoxynucleotides. J. Natl. Cancer Inst. 92: 34-41.
54. Baba, M., Iishi, H., and Tatsuta, M. 2000. In vivo electroporetic transfer of Bcl-2 antisense oligonucleotide inhibits the development of hepatocellular carcinoma in rats. Int. J. Cancer 85: 260-266.
55. Li, F. Z., Ackermann, E. J., Bennett, C. F., Rothermel, A. L., Plescia, J., Tognin, S., et al. 1999. Pleiotropic cell-division defects and apoptosis induced by interference with survivin function. Nat. Cell Biol. 1: 461-466.
56. Chen, J., Wu, W., Tahir, S. K., Kroeger, P. E., Rosenberg, S. H., Cowsert, L. M., et al. 2000. Down-regulation of survivin by antisense oligonucleotides increases apoptosis, inhibits cytokinesis and anchorage-independent growth. Neoplasia 2:235-241.
57. Grossman, D., McNiff, J. M., Li, F. Z., and Altieri, D. C. 1999. Expression and targeting of the apoptosis inhibitor, survivin, in human melanoma. J. Invest. Dermatol. 113:1076-1081.
58. Kanwar, J. R., Shen, W. P., Berg, R., and Krissansen, G. W. 2001. Effect of survivin antagonists on the growth of established tumors and B7.1 immunogene therapy. J. Natl. Cancer Inst. 93:1541-1552.
59. O'Connor, D. S., Schechner, J. S., Adida, C., Mesri, M., Rothermel, A. L., Li, F. Z., et al. 2000. Control of apoptosis during angiogenesis by survivin expression in endothelial cells. Amer. J. Pathol. 156:393-398.
60. Tran, J., Rak, J., Sheehan, C., Saibil, S. D., LaCasse, E., Korneluk, R. G., et al. 1999. Marked induction of the IAP family antiapoptotic proteins survivin and XIAP by VEGF in vascular endothelial cells. Biochem. Biophys. Res. Commun. 264: 781-788.
61. Papapetropoulos, A., Fulton, D., Mahboubi, K., Kalb, R. G., O'Connor, D. S., Li, F. Z., et al. 2000. Angiopoietin-1 inhibits endothelial cell apoptosis via the Akt/survivin pathway. J. Biol. Chem. 275: 9102-9105.
62. Tran, J. Master, Z., Yu, J. L., and Rak, J. Dumont D J. Kerbel R S. 2002. A role for survivin in chemoresistance of endothelial cells mediated by VEGF. Proc. Nat. Acad. Sci. USA 99: 4349-4354.
63. Mesri, M., Morales-Ruiz, M., Ackermann, E. J., Bennett, C. F., Pober, J. S., Sessa, W. C., and Altieri, D. C. 2001. Suppression of vascular endothelial growth factor-mediated endothelial cell protection by survivin targeting. Am. J. Path. 158: 1757-1765.
64. Olie, R. A., Simoes-Wust, A. P., Baumann, B., Leach, S. H., Fabbro, D., Stahel, R. A., et al. 2000. A novel antisense oligonucleotide targeting survivin expression induces apoptosis and sensitizes lung cancer cells to chemotherapy. Cancer Res. 60: 2805-2809.
65. Baguley, B. C., and Ching, L. M. 1997. Immunomodulatory actions of xanthenone anticancer agents. BioDrugs 8: 119-127.
66. Ruegg, C. Yilmaz, A., Bieler, G., Bamat, J., Chaubert, P., and Lejeune, F. J. 1998. Evidence for the involvement of endothelial cell integrin alphaVbeta3 in the disruption of the tumor vasculature induced by TNF and IFN-gamma. Nature Med. 4: 408-414.
67. Kanwar, J. R., Kanwar, R., Pandey S., Ching, L-M., and Krissansen, G. W. 2000. Vascular attack by 5,6-dimethylxanthenone-4-acetic acid combined with B7.1-mediated immunotherapy overcomes immune-resistance and leads to the eradication of large tumors. Cancer Res. 61: 1948-1956.
68. Siim, B. G., Lee, A. E., Shalal-Zwain, S., Pruijn, F. B., McKeage, M. J., and Wilson, W. R. 2003. Marked potentiation of the antitumor activity of chemotherapeutic drugs by the antivascular agent 5,6-dimethylxanthenone-4-acetic acid (DMXAA). Cancer Chemother. Pharmacol. 51: 43-52.
69. Kanwar, J., Berg, R., Lehnert, K., and Krissansen, G. W. 1999. Taking lessons from dendritic cells: Multiple xenogeneic ligands for leukocyte integrins have the potential to stimulate anti-tumor immunity. Gene Therapy 6: 1835-1844.
70. Sun, X., Leung, E., Kanwar, J. R., Lehnert, K., Wang, D., and Krissansen, G. W. 2001. Gene transfer of antisense hypoxia inducible factor-1α enhances the therapeutic efficacy of cancer immunotherapy. Gene Therapy 8: 638-645.
71. Sun, X., Kanwar, J. R., Leung, E., Lehnert, K., Wang, D., and Krissansen, G. W. 2001. Angiostatin enhances B7.1-mediated cancer immunotherapy independently of effects on vascular endothelial growth factor expression. Cancer Gene Ther. 8: 719-727.
72. Huang, X., Wong, M. K., Yi, H., Watkins, S., Laird, A. D., Wolf, S. F., and Gorelik, E. 2002. Combined therapy of local and metastatic 4T1 breast tumor in mice using SU6668, an inhibitor of angiogenic receptor tyrosine kinases, and the immunostimulator B7.2-IgG fusion protein. Cancer Res. 62: 5727-5735.
73. Sun, X., Leung, E., Kanwar, J. R., Lehnert, K., Wang, D., and Krissansen, G. W. Gene transfer of antisense hypoxia inducible factor-1α enhances the therapeutic efficacy of cancer immunotherapy. Gene Therapy 8: 638-645, 2001.
74. Kanwar, J. R., Shen, W. P., Berg, R., and Krissansen, G. W. Effect of survivin antagonists on the growth of established tumors and B7.1 immunogene therapy. J. Natl. Cancer Inst. 93:1541-1552, 2001.
75. O'Reilly M S, Holmgren L, Shing Y, et al. Angiostatin: a novel angiogenesis inhibitor that mediates the suppression of metastases by a Lewis lung carcinoma. Cell. 1994; 79:315-328.
76. O'Reilly M S, Holmgren L, Chen C, et al. Angiostatin induces and sustains dormancy of human primary tumours in mice. Nat Med. 1996; 2:689-692.
77. Boehm T, Folkman J, Browder T, et al. Antiangiogenic therapy of experimental cancer does not induce acquired drug-resistance. Nature 1997; 390:404-407.
78. Moser T L, Stack M S, Asplin I, et al. Angiostatin binds ATP synthase on the surface of human endothelial cells. Proc Natl Acad Sci USA 1999; 96:2811-2816.
79. Holmgren L, O'Reilly M S, Folkman J. Dormancy of micrometastases-balanced proliferation and apoptosis in the presence of angiogenesis suppression. Nat Med. 1995; 1: 149-153.
80. Claesson-Welsh L, Welsh M, Ito N, et al. Angiostatin induces endothelial cell apoptosis and activation of focal adhesion kinase independently of the integrin-binding motif RGD. Proc Natl Acad Sci USA. 1998; 95:5579-5583.
81. Kirsch M, Strasser J, Allender R, et al. Angiostatin suppresses malignant glioma growth in vivo. Cancer Res. 1998; 58:4654-4659.
82. Joe Y A, Hong Y K, Chung D S, et al. Inhibition of human malignant glioma growth in vivo by human recombinant plasminogen kringles 1-3. Int J Cancer 1999; 82:694-699.
83. Redlitz A, Daum G, Sage E H. Angiostatin diminishes activation of the mitogen-activated protein kinase ERK-1 and ERK-2 in human dermal microvascular endothelial cells. J Vasc Res. 1999; 36:28-34.
84. Ito H, Rovira I I, Bloom M L, et al. Endothelial progenitor cells as putative targets for angiostatin. Cancer Res. 1999; 59:5875-5877.
85. Sun, X., Kanwar, J. R., Leung, E., Lehnert, K., Wang, D., and Krissansen, G. W. Angiostatin enhances B7.1-mediated cancer immunotherapy independently of effects on vascular endothelial growth factor expression. Cancer Gene Ther. 8: 719-727, 2001.
86. Xu, R., Sun, X., Chan, D., Li, H., Tse, L-Y., Xu, S., Xiao, W., Kung, H., Krissansen, G. W., and Fan, S-T. Long-term expression of angiostatin suppresses metastatic liver cancer in mice. Hepatol. 37:1451-60, 2003.

1. A method of treating tumors in a mammal, the method comprising at least the step of administering an effective amount of a HIF inhibiting agent together with an effective amount of at least one antiangiogenic agent.

2. A method as claimed in claim 1, wherein the HIF inhibiting agent is antisense HIF-1.

3. A method as claimed in claim 2, wherein the antisense HIF-1 is antisense HIF-1α.

4. A method as claimed claim 1, wherein the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

5. A method as claimed in claim 2, wherein antisense HIF-1α is provided by a vector adapted to produce antisense HIF-1α in use.

6. A method as claimed in claim 4, wherein an agent capable of increasing VHL in a tumor is a vector adapted to express VHL in use.

7. A method as claimed in claim 4, an agent capable of increasing VHL in a tumor is one adapted to over-express native VHL within the tumor.

8. A method as claimed claim 4, wherein antisense survivin is provided by a vector adapted to produce antisense survivin in use.

9. A method as claimed in claim 4, wherein VEGF blocking peptide is provided by a vector adapted to express VEGF blocking peptide in use.

10. A method as claimed in claim 4, wherein angiostatin is provided by a vector adapted to express angiostatin in use.

11. A method as claimed in claim 4, wherein endostatin is provided by a vector adapted to express endostatin in use.

12. A method as claimed in claim 4, wherein antisense HIF-1α, an agent capable of increasing VHL, antisense survivin, VEGF blocking peptide, angiostatin, or endostatin is provided by a nucleic acid vector.

13. A method as claimed in claim 4, wherein antisense HIF-1α, an agent capable of increasing VHL, antisense survivin, VEGF blocking peptide, angiostatin, or endostatin is provided by a viral vector comprising nucleic acid in a viral capsid.

14. A method as claimed in claim 2, wherein the antisense HIF-1α, and one or more antiangiogenic agents are administered intratumorally.

15. A method as claimed in claim 2, wherein the antisense HIF-1α, and one or more antiangiogenic agents are administered intraperitoneally, parenterally, or systemically.

16. A method as claimed in claim 3, wherein the antisense HIF-1α and one or more antiangiogenic agents are coadministered.

17. A method as claimed in claim 3, wherein the antisense HIF-1α and one or more antiangiogenic agents are administered sequentially, in any order.

18. A method of treating a tumor in an animal comprising at least the step of administering to said animal antisense HIF-1α with endostatin and/or VEGF blocking protein.

19. A method as claimed in claim 18, wherein antisense HIF-1α is administered in the form of a vector adapted to produce antisense HIF-1α in use.

20. A method as claimed in claim 18, wherein VEGF blocking protein and endostatin are co-administered.

21. A method as claimed in claim 18, wherein the administration of antisense HIF-1α and the co-administration of endostatin and VEGF blocking protein, proceed sequentially.

22. A method as claimed in claim 18, wherein endostatin and VEGF blocking protein are administered subcutaneously.

23. A method of treating a tumor in an animal comprising at least the steps of administering to said animal antisense HIF-1α and over-expressing VHL in the tumor.

24. A method as claimed in claim 23, wherein over-expression of VHL occurs via administering a vector adapted to express VHL in use.

25. A method as claimed in claim 23, wherein antisense HIF-1α is administered in the form of a vector adapted to produce antisense HIF-1α in use.

26. A method as claimed in claim 23, wherein administration of the vector adapted to express VHL occurs first, followed by administration of the vector adapted to express antisense HIF-1α.

27. A method of treating a tumor in an animal comprising at least the steps of administering to said animal antisense HIF-1α and angiostatin.

28. A method as claimed in claim 27, wherein antisense HIF-1α is administered in the form of a vector adapted to produce antisense HIF-1α in use.

29. A method as claimed in claim 27, wherein angiostatin is administered in the form of a vector adapted to express angiostatin in use.

30. A method as claimed in claim 29, wherein the vector adapted to express angiostatin in use is administered first, followed by administration of the vector adapted to produce antisense HIF-1α in use.

31. A method of enhancing tumor cell apoptosis in an animal, the method comprising at least the step of administering an effective amount of antisense HIF-1α together with an effective amount of at least one antiangiogenic agent.

32. A method as claimed in claim 31, wherein the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

33. A method of inhibiting tumor angiogenesis in an animal, the method comprising at least the step of administering an effective amount of antisense HIF-1α together with an effective amount of at least one antiangiogenic agent.

34. A method as claimed in claim 33, wherein the antiangiogenic agent is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

35. A composition comprising antisense HIF-1α, or a vector adapted to produce antisense HIF-1α in use, together with one or more antiangiogenic agents and optionally one or more pharmaceutically acceptable excipients or carriers.

36. A composition as claimed in claim 35, wherein the antiangiogeneic agents are selected from the group comprising endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

37. A composition as claimed in claim 35, wherein the composition is suitable for intratumoral administration.

38. A composition as claimed in claim 35, wherein the composition is suitable for intraperitoneal administration.

39. A composition as claimed in claim 35, wherein the composition is suitable for systemic administration.

40. A composition as claimed in claim 35, wherein the composition is suitable for subcutaneous administration.

41. A composition combining (i) antisense HIF-1α, or a vector adapted to produce antisense HIF-1α and (ii) one or more antiangiogenic agents, wherein the combination of (i) and (ii) is adapted for sequential administration to a mammal.

42. The use of antisense HIF-1α, or a vector adapted to produce antisense HIF-1α, and one or more antiangiogenic agents in the manufacture of a medicament for enhancing tumor cell apoptosis, inhibiting tumor angiogenesis, or for tumor treatment in an animal.

43. The use as claimed in claim 42, wherein the antiangiogenic agents are selected from the group comprising endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

44. A method of systemically treating tumors in a mammal, comprising at least, in any order, the steps of:
(a) administering a systemically effective amount of an HIF-1 inhibiting agent and
(b) administering a systemically effective amount of an antiangiogenic agent.

45. A method as claimed in claim 44, wherein the HIF-1 inhibiting agent and the antiangiogenic agent are administered by subcutaneous injection, together with suitable carriers.

46. A method as claimed in claim 45, wherein the HIF-1 inhibiting agent that is subcutaneously administered is selected from any one or more of HIF-1 antagonists including cellular ligands, and cell permeable agents that antagonises HIF-1 expression and function such as cell-permeable VHL, cell-permeable dominant-negative HIF-1 peptides, and antisense HIF-1 polynucleotides.

47. A method as claimed in claim 45, wherein the antiangiogenic agent that is subcutaneously administered is selected from any one or more of endostatin, angiostatin, VEGF blocking peptide or a mimetic thereof, or another agent capable of blocking the expression or function of VEGF, VHL, an agent capable of increasing VHL in a tumor, a VHL function mimicking agent, antisense survivin, or other agent capable of blocking the expression or function of survivin.

48. A method as claimed in claim 44, wherein step (a) and step (b) are separate sequential steps in any order.

49. A method as claimed in claim 44, wherein step (a) and step (b) are unitary and the agents are co-administered.

50. A method of treating a tumor in an animal comprising at least the steps of administering to said animal antisense HIF-1α and antisense survivin.

51. A method as claimed in claim 50, wherein antisense HIF-1α and antisense survivin are administered in the form of a vector adapted to produce antisense HIF-1α in use.
